(19) United States
(12) Patent Application Publication
(10) Pub. No.: US 2008/0132698 A1
(43) Pub. Date: Jun. 5, 2008

(54) Use of N-oxide compounds in coupling reactions

(75) Inventors: Keith Fagnou, Ottawa (CA); Jean-Philippe Leclerc, Gatineau (CA); Louis-Charles Campeau, Gatineau (CA); David R. Stuart, Ottawa (CA)

Correspondence Address: MCCARTER & ENGLISH LLP;CITYPLACE I, 185 ASYLUM STREET, HARTFORD, CT, 06103, US

(73) Assignee: University of Ottawa

(21) Appl. No.: 11/606,499

(22) Filed: Nov. 30, 2006

(51) Int. Cl. C07D 237/06; C07D 241/10; C07D 239/24; C07D 241/36

(52) U.S. Cl. 544/224; 544/336; 544/242; 544/353

(57) ABSTRACT

Metal-catalyzed coupling process comprising reacting a compound of general formula 1 with a compound A-X, to obtain a compound of general formula 2, which may further be converted to a compound of general formula 3

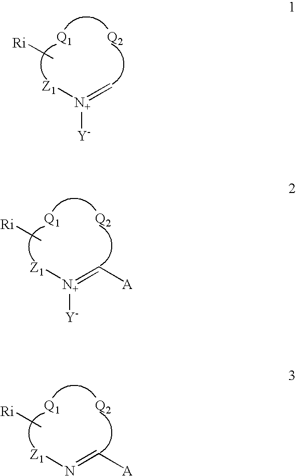

### FIELD OF THE INVENTION

The invention relates generally to coupling reactions. In particular, the invention relates to the use of N-oxides in metal-catalyzed coupling reactions.

### BACKGROUND OF THE INVENTION

While transition metal-catalyzed coupling reactions between a wide range of halides and organometallics have been successful,¹ such coupling reactions between some substrate classes still pose significant challenges. This is the case for example between coupling reactions between some halides and many metalloazines and azines. Indeed, frequent instability and difficult synthesis of 2-pyridylorganometallics severely limits their use. Examples of coupling reactions between 2-halopyridines and aryl boronic acids are well known in the art.² However, the inherent instability of 2-pyridyl boronic acid makes successful couplings involving them rare.³ Given the importance in materials⁴ and medicinal chemistry of reaction products of such couplings,⁵ there is a need for the development of improved processes for the preparation of these products. In particular, a readily available, bench-stable replacement for 2-pyridyl organometallics for use in these coupling reactions would present a significant advantage.

In recent years, direct arylation has emerged as an attractive alternative to some typical coupling reactions.⁶ In direct arylation, one of the preactivated coupling partners (typically the organometallic species) is replaced by an unfunctionalized arene. Consistent with an electrophilic aromatic substitution (S_{E}Ar) pathway, thus electron-rich heterocyclic arenes have been featured prominently in recent developments.⁷ While some simple arenes can now be used,^{8,9} direct arylation reactions with n-electrondeficient heteroarenes, such as pyridine, remain a challenging goal.¹⁰

Also, palladium-catalyzed cross-coupling reactions in biaryl synthesis are known in the art.¹¹ These reactions are largely linked to, and limited by, the synthetic and commercial availability of organometallic reagents involved including aryl boronic acids. In addition, there is a significant cost associated with most of these reagents. There thus remain important classes of aryl organometallic that are very challenging to prepare and/or to use in coupling reactions including electron deficient nitrogen-containing heterocycles.¹ The importance in medicinal and materials sciences¹² of building blocks, products of couplings between aryl organometallics and nitrogen-containing heterocycles has prompted continued methodological efforts and two recent reports by Fu¹³ and Buchwald¹⁴ highlight the importance of this goal.

The most problematic subset of organometallic reagents are those bearing the organometallic adjacent to a nitrogen atom. The problem is even more severe when two nitrogen atoms are present in the aromatic ring as illustrated in FIG. 1. These organometallics are difficult to prepare, unstable, and generally decompose under coupling reaction conditions. While some are commercially available, the price reflects both their value and the challenge associated with their preparation.¹⁵

### SUMMARY OF THE INVENTION

The inventors of the present application have now discovered that the use of N-oxides in metal-catalysed coupling reactions presents significant advantage over the use of organometallics. Indeed, pyridine N-oxides for example are commercially available or easily prepared,¹⁶ and are inexpensive. They can be used as bench-stable replacements for problematic 2-metalla-pyridines. Direct arylation of pyridine N-oxides with a wide range of aryl bromides occurs in excellent yields with complete selectivity for the 2-position. The inventors have also shown that a wide range of N-oxides and can be easily prepared and used in the coupling process according to the invention.

The products obtained from the coupling process according to the invention can be used in the preparation of various compounds having therapeutic or industrial application. In particular, the products can be converted to corresponding free amine products.

The invention thus provides according to a first aspect for a coupling process comprising reacting a compound of general formula 1 with a compound of general formula A-X, in the presence of a first metal catalyst, to obtain a compound of general formula 2

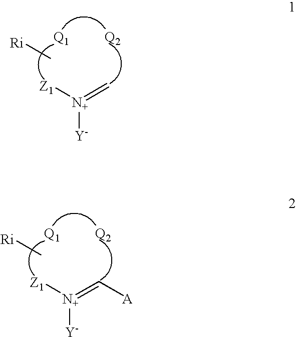

wherein Y is O or S; Z₁ is C, N, O or S, and is optionally substituted when it is C or N; Q₁, Q₂ and A each represents a chemical group which is independently linear or branched, saturated or unsaturated, aromatic, cyclic, bicyclic or biaryl the chemical group containing or not containing a hetero atom which is N, O, S or a halogen atom; ( denotes a chemical bond that is present or absent; Ri represents at least one substituent that is linear or branched, saturated or unsaturated, aromatic, cyclic or bicyclic, the substituent containing or not containing a hetero atom, with the proviso that N, Z₁, Q₁, Q₂ and C form a ring, optionally Ri together with the ring forms a bicyclic or biaryl group; X represents a leaving group; and C directly attached to N⁺ in 1 is not substituted.

According to a second aspect, the invention provides for a coupling process comprising reacting a compound of general formula 2A with a compound of general formula A′-X, in the presence of a first metal catalyst, to obtain a compound of general formula 4

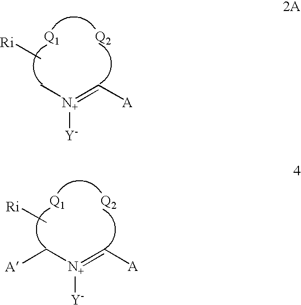

wherein Y is O or S; Q₁, Q₂, A and A′ each represents a chemical group which is independently linear or branched, saturated or unsaturated, aromatic, cyclic, bicyclic or biaryl, the chemical group containing or not containing a hetero atom which is N, O, S or a halogen atom; ( denotes a chemical bond that is present or absent; Ri represents at least one substituent that is linear or branched, saturated or unsaturated, aromatic, cyclic or bicyclic, the substituent containing or not containing a hetero atom, with the proviso that N, Q₁, Q₂ and the two carbon atoms form a ring, optionally Ri together with the ring forms a bicyclic or biaryl group; X represents a leaving group; and the other C directly attached to N⁺ and not bearing substituent A in 2A is not substituted.

According to a third aspect, the invention provides for a coupling process comprising reacting a compound of general formula 6 with a compound of general formula 29, in the presence of a first metal catalyst, to obtain a compound of general formula 7

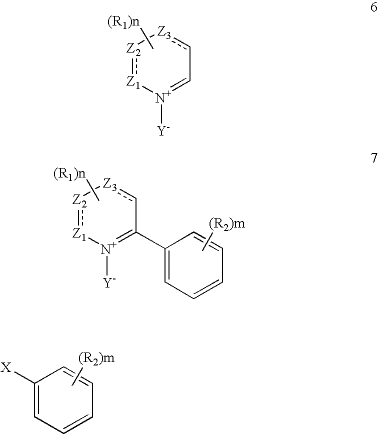

wherein Y is O or S; Z₁, Z₂ and Z₃ are each independently C, N, O or S, and are each independently optionally substituted when they are C or N; R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group; — denotes a chemical bond that is present or absent; n is 0, 1, 2, 3 or 4; m is 0, 1, 2, 3, 4 or 5; X is a leaving group; and C directly attached to N⁺ in 6 is not substituted.

According to a fourth aspect, the invention provides for a coupling process comprising reacting a compound of general formula 7A with a compound of general formula 30, in the presence of a first metal catalyst, to obtain a compound of general formula 9

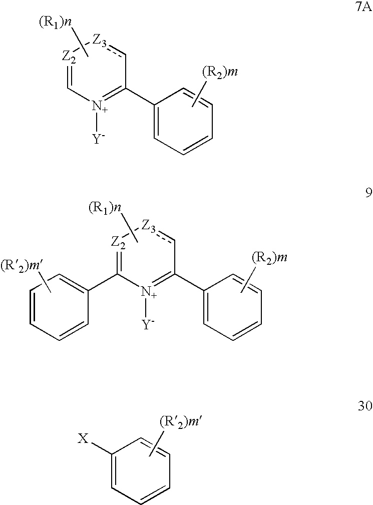

wherein Y is O or S; Z₂ and Z₃ are each independently C, N, O or S, and are each independently optionally substituted when they are C or N; R₁, R₂ and R′₂ are each independently linear or branched, saturated or unsaturated, aromatic, cyclic, bicyclic, contains or not contains a hetero atom which is N, O, S or a halogen atom, optionally R₁, R₂ or R′₂ together with the ring to which it is attached forms a bicyclic or biaryl group; n is 0, 1, 2 or 3; m and m′ are each independently 0, 1, 2, 3, 4 or 5; X is a leaving group; and the other C directly attached to N⁺ in 7A is not substituted.

According to a fifth aspect, the invention provides for a coupling process comprising reacting a compound of general formula 6′ with a compound of general formula 29, in the presence of a first metal catalyst, to attain a compound of general formula 7′

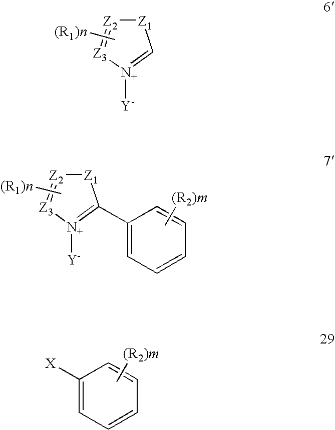

wherein Y is O or S; Z₁, Z₂ and Z₃ are each independently C, N, O or S, and are each independently optionally substituted when they are C or N; R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group; n is 0, 1, 2, 3 or 4; m is 0, 1, 2, 3, 4 or 5; X is a leaving group; and C directly attached to N⁺ in 6′ is not substituted.

According to a sixth aspect, the invention provides for a coupling process comprising reacting a compound of general formula 7A′ with a compound of general formula 30, in the presence of a first metal catalyst, to obtain a compound of general formula 9′

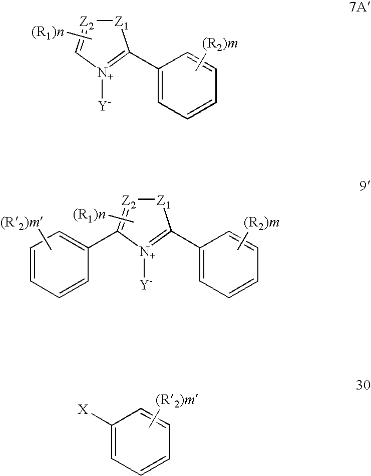

wherein Y is O or S; Z₁ and Z₂ are each independently C, N, O or S, and are each independently optionally substituted when they are C or N; R₁, R₂ and R′₂ are each independently linear or branched, saturated or unsaturated, aromatic, cyclic, bicyclic, contains or not contains a hetero atom which is N, O, S or a halogen atom, optionally R₁, R₂ or R′₂ together with the ring to which it is attached forms a bicyclic or biaryl group; n is 0, 1, 2 or 3; m and m′ are each independently 0, 1, 2, 3, 4 or 5; X is a leaving group; and the other C directly attached to N⁺ in 7A′ is not substituted.

According to a seventh aspect, the invention provides for a coupling process comprising reacting a compound of general formula 11 with a compound of general formula 29, in the presence of a first metal catalyst, to obtain a compound of general formula 12

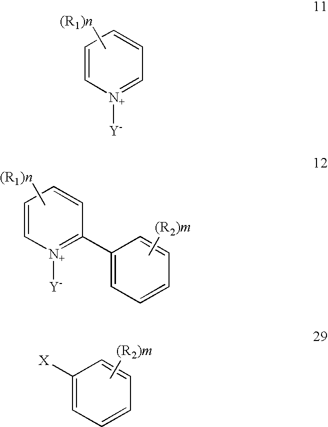

wherein Y is O or S; R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group; n is 0, 1, 2, 3 or 4; m is 0, 1, 2, 3, 4 or 5; X is a leaving group; and at least one C directly attached to N⁺ in 11 is not substituted.

According to an eighth aspect, the invention provides for a coupling process comprising reacting a compound of general formula 12 with a compound of general formula 30, in the presence of a first metal catalyst, to obtain a compound of general formula 14

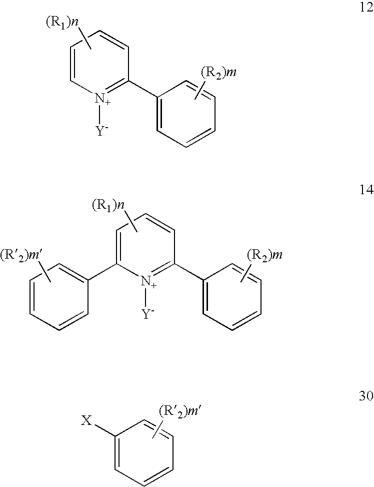

wherein Y is O or S; R₁, R₂ and R′₂ are each independently linear or branched, saturated or unsaturated, aromatic, cyclic, bicyclic, contains or not contains a hetero atom which is N, O, S or a halogen atom, optionally R₁, R₂ or R′₂ together with the ring to which it is attached forms a bicyctic or biaryl group; n is 0, 1, 2 or 3; m and m′ are each independently 0, 1, 2, 3, 4 or 5; X is a leaving group; and the other C directly attached to N⁺ in 12 is not substituted.

According to a ninth aspect, the invention provides for a coupling process comprising reacting a compound of general formula 16 with a compound of general formula 29, in the presence of a first metal catalyst, to obtain a compound of general formula 17

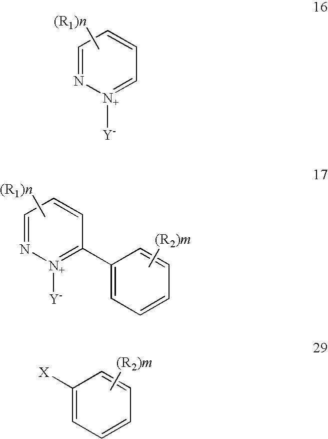

wherein Y is O or S; R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group; n is 0, 1, 2, 3 or 4; m is 0, 1, 2, 3, 4 or 5; X is a leaving group; and C directly attached to N⁺ in 16 is not substituted.

According to a tenth aspect, the invention provides for a coupling process comprising reacting a compound of general formula 19 with a compound of general formula 29, in the presence of a first metal catalyst, to obtain a compound of general formula 20

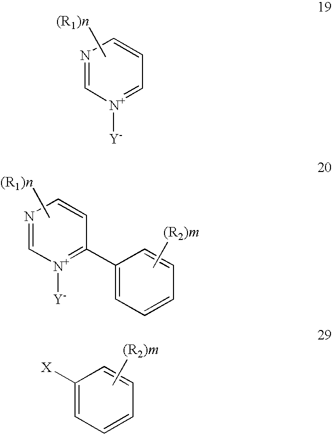

wherein Y is O or S; R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group; n is 0, 1, 2, 3 or 4; m is 0, 1, 2, 3, 4 or 5; X is a leaving group; and C directly attached to N⁺ in 19 is not substituted.

According to an eleventh aspect, the invention provides for a coupling process comprising reacting a compound of general formula 20 with a compound of general formula 30, in the presence of a first metal catalyst, to obtain a compound of general formula 22

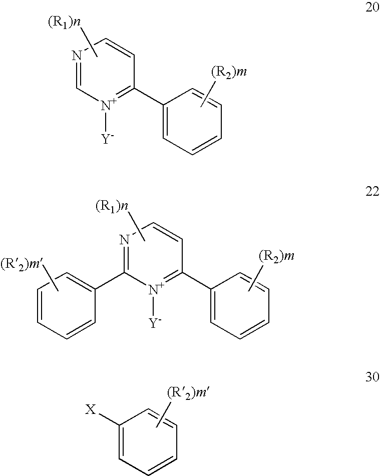

wherein Y is O or S; R₁, R₂ and R′₂ are each independently linear or branched, saturated or unsaturated, aromatic, cyclic, bicyclic, contains or not contains a hetero atom which is N, O, S or a halogen atom, optionally R₁, R₂ or R′₂ together with the ring to which it is attached forms a bicyclic or biaryl group; n is 0, 1, 2 or 3; m and m′ are each independently 0, 1, 2, 3, 4 or 5; X is a leaving group; and the other C directly attached to N⁺ in 20 is not substituted.

According to a twelfth aspect, the invention provides for a coupling process comprising reacting a compound of general formula 24 with a compound of general formula 29, in the presence of a first metal catalyst, to obtain a compound of general formula 25

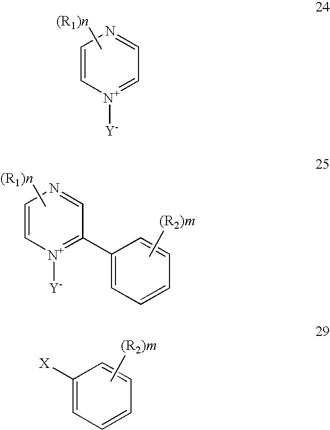

wherein Y is O or S; R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group; n is 0, 1, 2, 3 or 4; m is 0, 1, 2, 3, 4 or 5; X is a leaving group; and at least one C directly attached to N⁺ in 24 is not substituted.

According to a thirteenth aspect, the invention provides for a coupling process comprising reacting a compound of general formula 25 with a compound of general formula 30, in the presence of a first metal catalyst, to obtain a compound of general formula 27

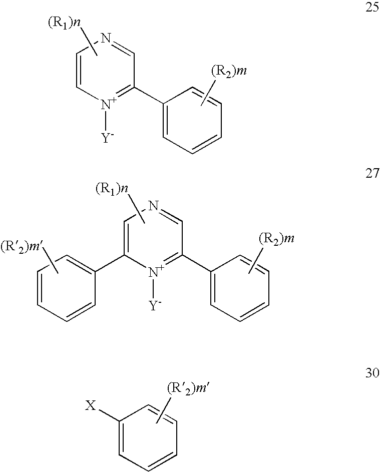

wherein Y is O or S; R₁, R₂ and R′₂ are each independently linear or branched, saturated or unsaturated, aromatic, cyclic, bicyclic, contains or not contains a hetero atom which is N, O, S or a halogen atom, optionally R_{1, R}₂ or R′₂ together with the ring to which it is attached forms a bicyclic or biaryl group; n is 0, 1, 2 or 3; m and m′ are each independently 0, 1, 2, 3, 4 or 5; X is a leaving group; and the other C directly attached to N⁺ in 25 is not substituted.

In embodiments of the above aspects of the invention, the reaction takes place in the presence of a metal salt which may be a Cu salt or other suitable salts known in the art. The metal salt can include CuCN, CuCl, CuBr or CuI, and is used in an amount of about 1 to 15 mol %, preferably about 10 mol %, based on compound A-X or A′-X. In further embodiments, the reaction also takes place in the presence of a base which may include K₂CO₃, NaOH, KOH or K₃PO₄. The base is used in an amount of about 1 to 4 equivalent, preferably about 2 equivalent based on compound A-X or A′-X. The temperature of the reaction can be about 80 to 130° C. or preferably about 110° C.

The first metal catalyst in the above aspects of the invention can be a Pd catalyst or other suitable catalysts known in the art. The first metal catalyst may include Pd(OAc)₂, PdCl₂, PdBr₂ or PdI₂, and is used in an amount of about 2 to 10 mol %, preferably about 5 mol %, based on the compound to be coupled with (A-X or A′-X).

The reaction in the process according to the above aspects may take place in the presence of an organic solvent which is an aromatic solvent, dioxane, mesitylene, N,N-dimethylacetamide, N,N-dimethylformamide, N-methylpyrrolidinone, tetrahydrofuran, dichloromethane, ether or a mixture thereof. Optionally, the solvent may be benzene, toluene, dioxane or a mixture thereof.

The reaction can also take place in the presence of a P-containing ligand carbene which is PCy₃, Pt-Bu₂Me, Pt-Bu₃-HBF₄ or PR₃, wherein R is alkyl or aryl; or an N-heterocyclic compound with a ligand which is IMes, SIMes, IPr or SIPr. The P-containing ligand or the N-heterocyclic compound is used in an amount of about 10 to 20 mol %, preferably about 15 mol %, based on compound A-X or A′-X. Optionally, an additive may be used, which is capable of overcoming the poisoning effects of N-oxides substrates. The additive may be an Ag salt or other suitable salts known in the art, which is Ag₂CO₃, AgOTf, AgSbF₆, AgPF₆ or AgBF₄. The additive is used in an amount of about 0.1 to 4 equivalent, preferably about 2 equivalent, based on compound A-X or A′-X.

In the process according to the invention, an equivalent amount of about 1 to 6, preferably about 1 to 4, of the starting material, based on the compound to be coupled with (A-X or A′-X), is used. The reaction time in the process according to the invention may vary from about 5 to 30 hours, generally it may vary from about 8 to 16 hours. The substitution is regioselective to a carbon atom attached to N⁺.

According to a fourteenth aspect of the invention, the product obtained from the coupling process may further be converted to another product, in the presence of a second metal catalyst to yield compounds listed below:

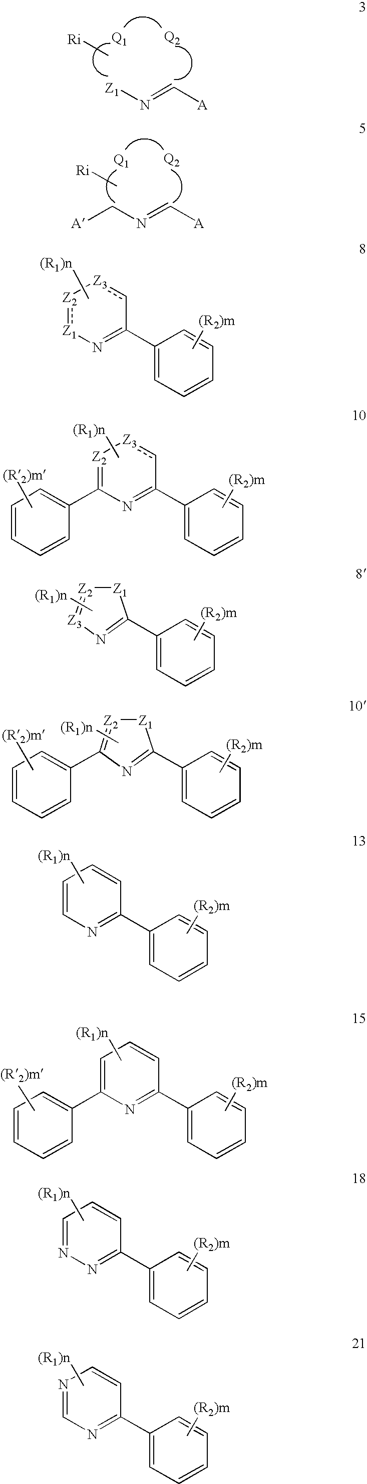

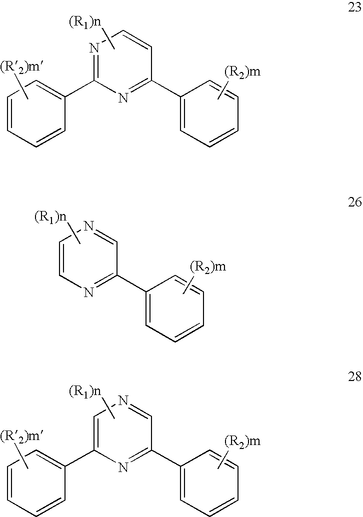

The second metal catalyst in the above further reaction may be a hydrogenation catalyst comprising Pd, Pt, Rh, Ir or Rn. Optionally, an organic salt such as HCOONH₄ or any other suitable organic salt, may be used. A gas such as H₂ may also be used. The reaction can take place in the presence of an organic solvent which is MeOH, EtOH, iPrOH, EtOAc, THF, acetone or a mixture thereof. Optionally, the solvent may be NH₄OH, EtOaC, THF, acetone or a mixture thereof. The reaction temperature may vary from about 15 to 30° C., preferably about 25° C.

Compounds obtained in the process of the invention may be used in the preparation of target compounds of therapeutic or industrial value.

### BRIEF DESCRIPTION OF THE DRAWINGS

FIG. 1 presents organometallic reagents known in the art.

FIG. 2 presents N-oxides used in the coupling process according to the invention.

FIG. 3 presents a general reaction scheme of the coupling process according to the invention.

FIGS. 4, 4′ and 5 to 8 present reaction schemes of aspects of the process according to the invention.

FIGS. 9-11 illustrate uses of the N-oxides according to the invention in the preparation of a wide range of compounds.

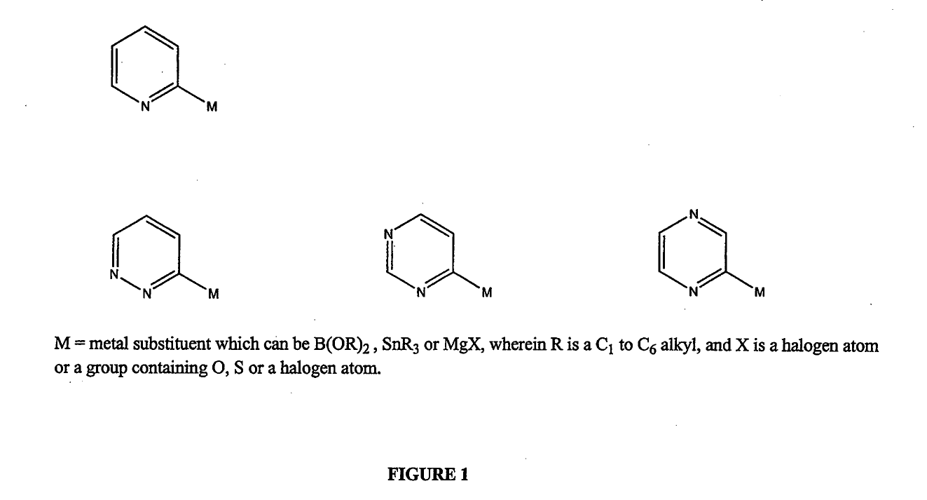

### DETAILED DESCRIPTION OF THE INVENTION

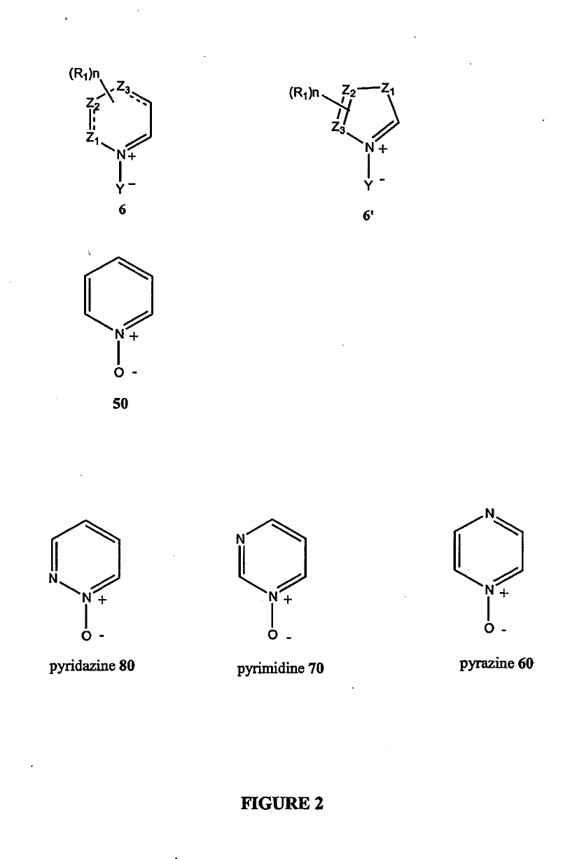

In FIG. 2 are represented pyridine, pyridazine, pyrimidine and pyrazine N-oxides (6, 6′, 50, 60, 70, 80) that are used as replacements for organometallic reagents in coupling reactions, in particular in the preparation of biaryl compounds.

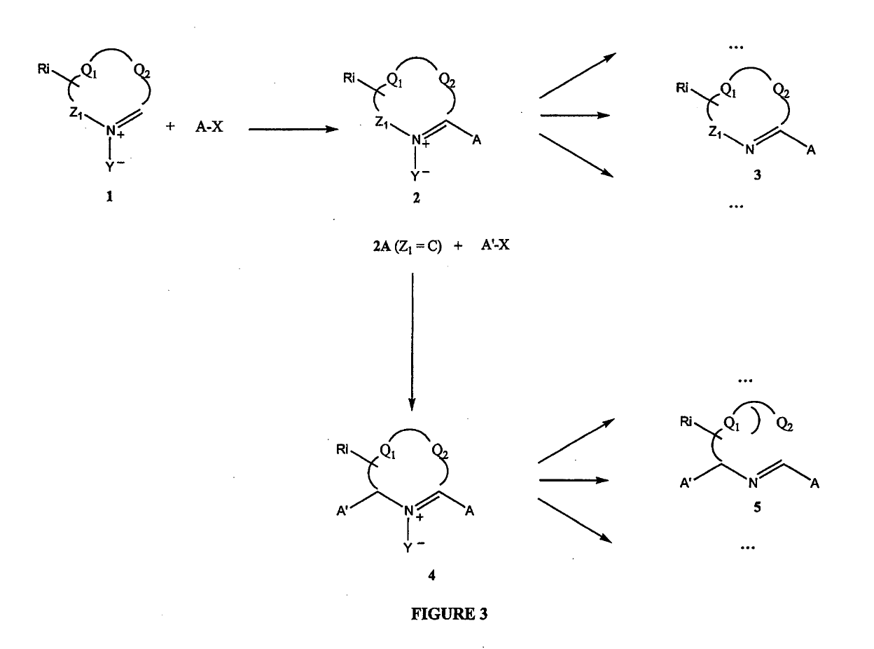

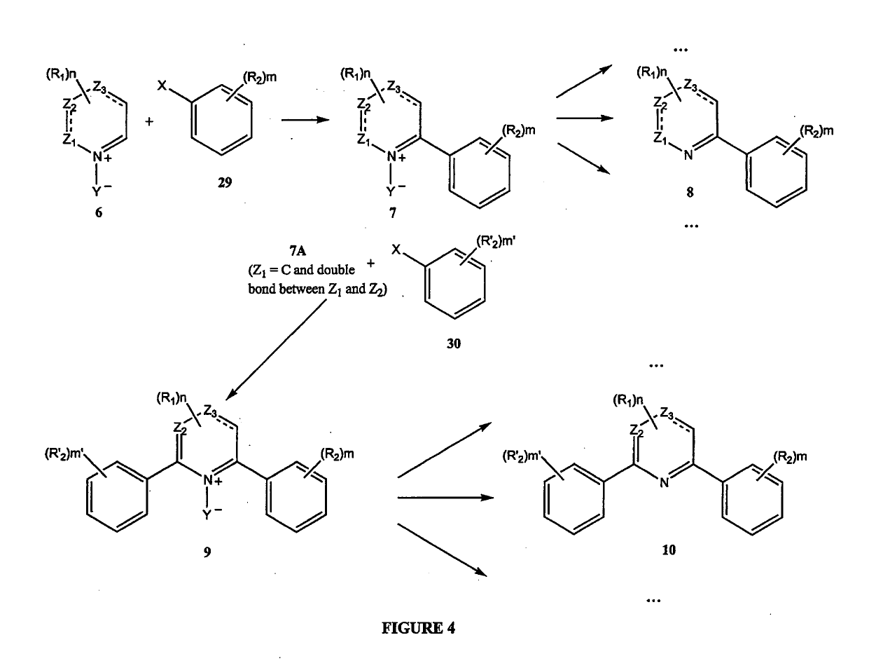

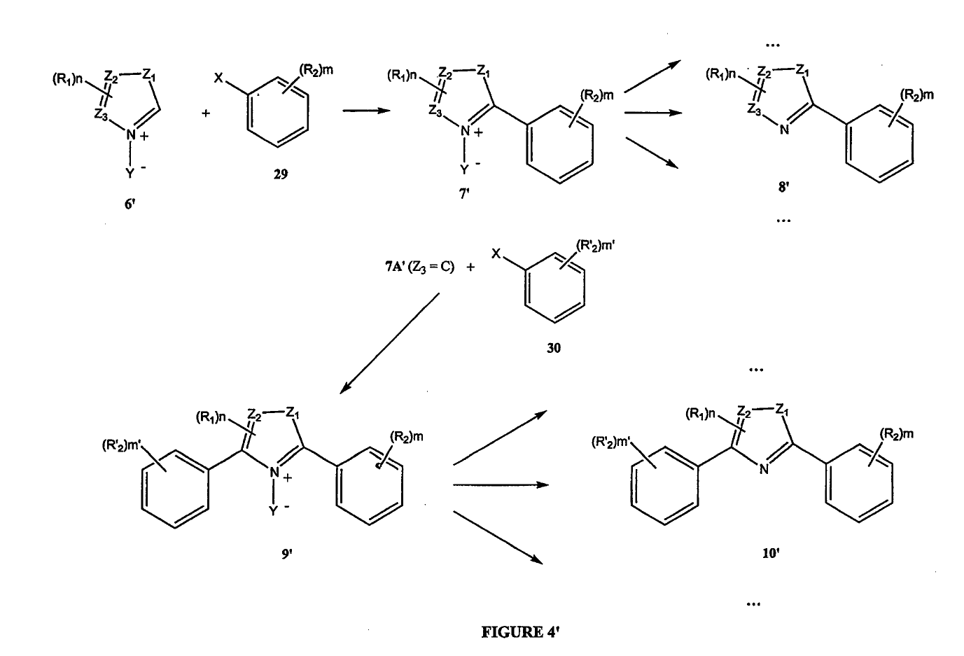

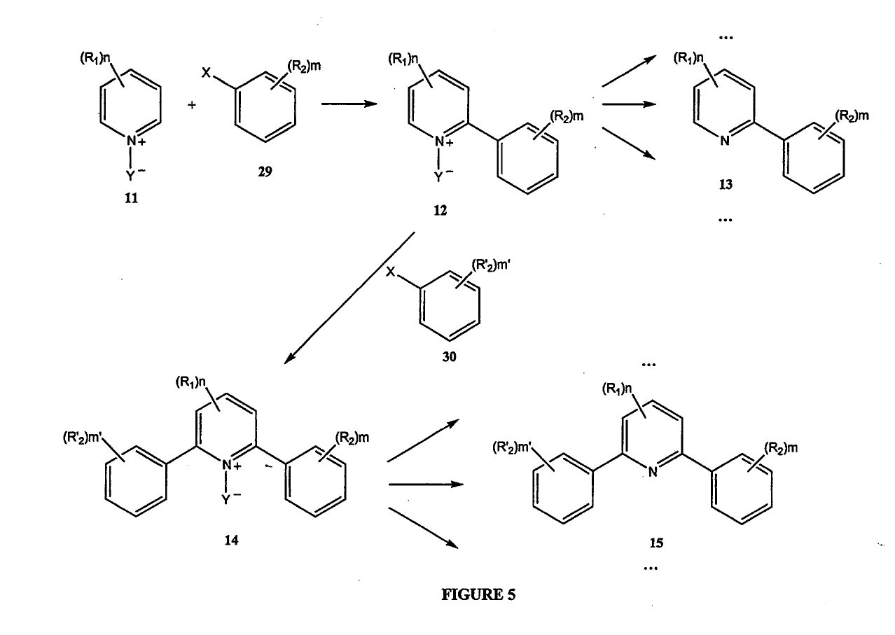

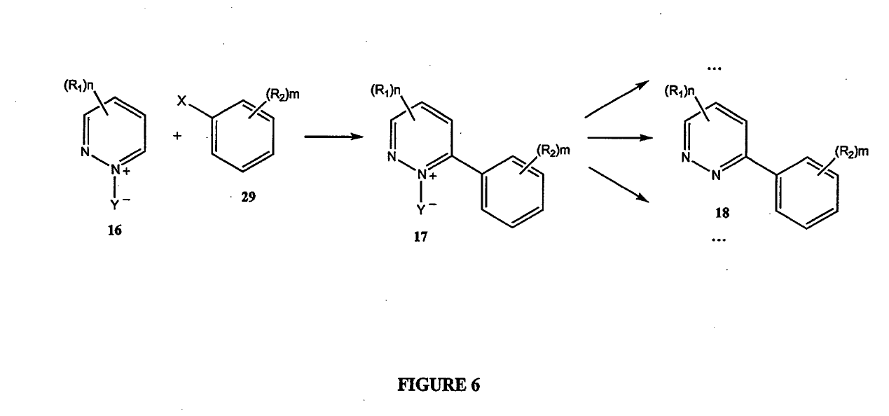

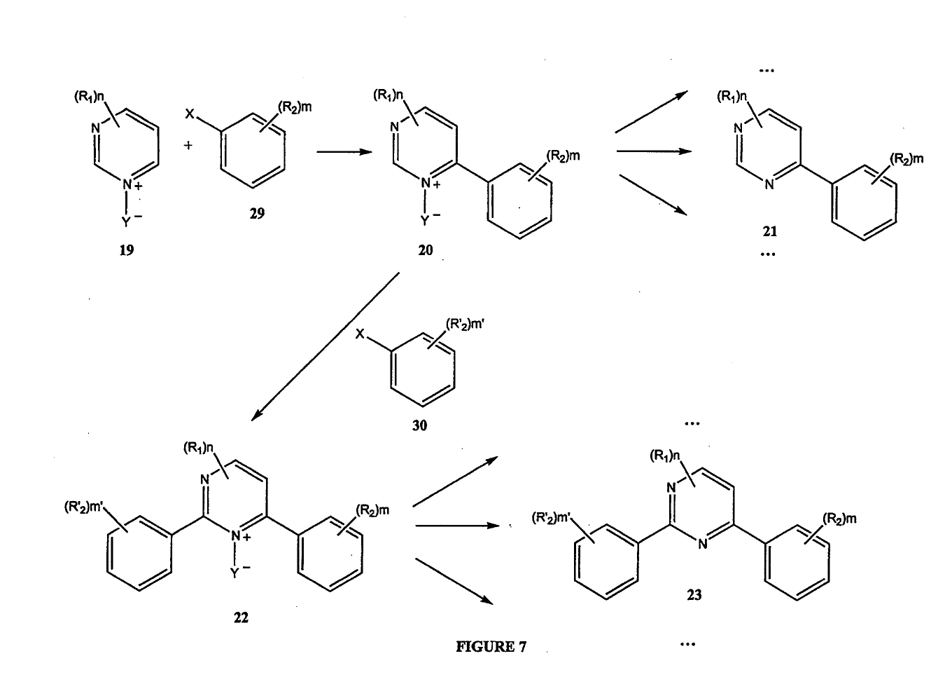

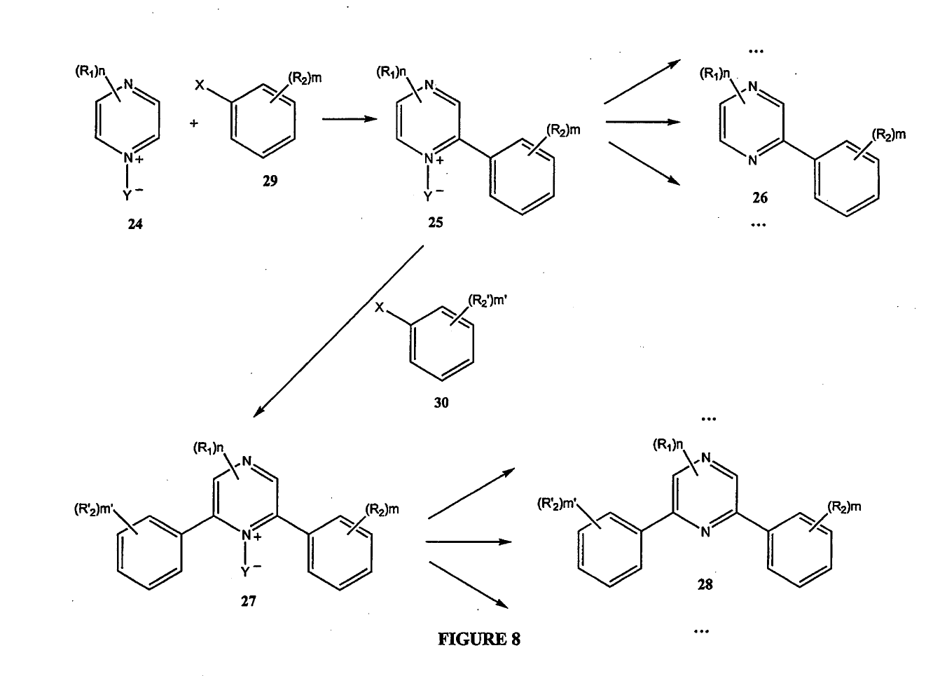

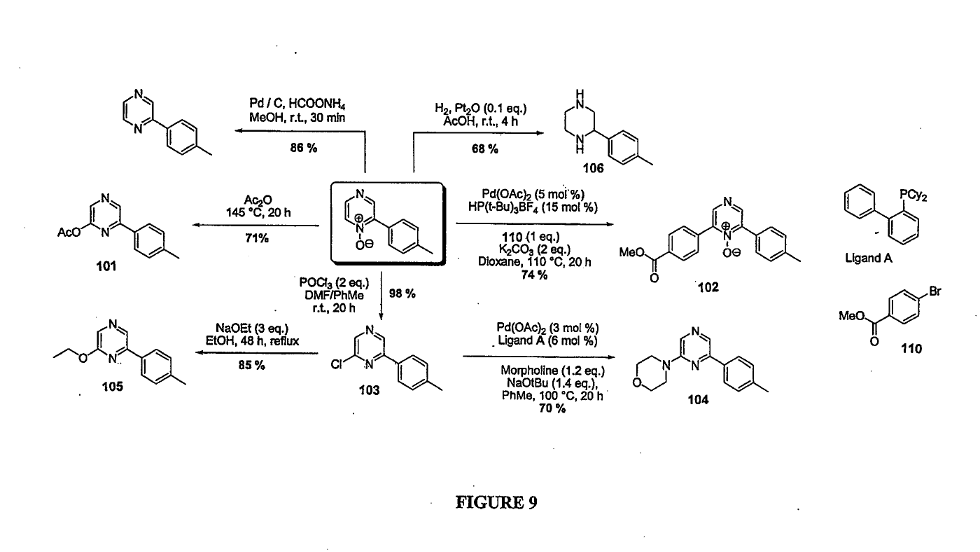

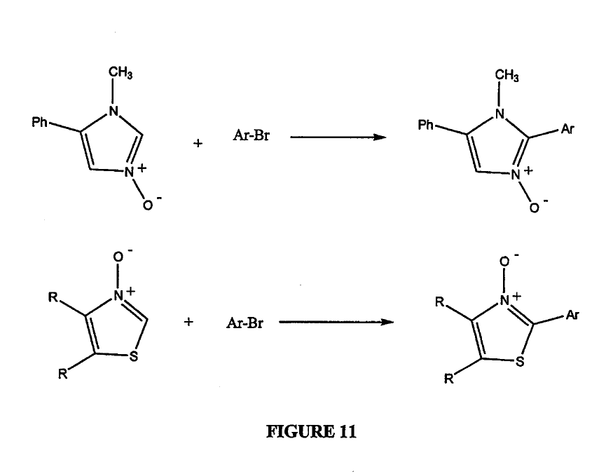

FIGS. 3 to 11 illustrate aspects of the process according to the invention.

Reaction development was carried out with pyridine N-oxide and 4-bromotoluene. Palladium acetate in combination with tri-tert-butylphosphine (added to the reaction mixture as the commercially available and air-stable HBF₄ salt) was used as metal-ligand combination. Potassium carbonate was used as base, and toluene was used as solvent. Other suitable solvents include dioxane, mesitylene, N,N-dimethylacetamide, tetrahydrofuran, dichloromethane and ether. The reactions were run under quite concentrated conditions (0.3 M), with 2-4 equiv of pyridine N-oxide. Under these conditions (4-bromotoluene, 2-4 equiv of pyridine N-oxide, 5 mol % of Pd—(OAc)₂, 15 mol % of Pt-Bu₃.HBF₄, 2 equiv of K₂CO₃ in toluene at 110° C.), 2-tolylpyridine N-oxide was obtained in 91% isolated yield exclusively as one regloisomer (Table 1, entry 1).

While 4 equiv of the N-oxide are not required, under these conditions, a decrease to 1 equiv leads to diminished yields (entries 2-8). When 1 equiv of pyridine N-oxide was used, greater than 95% of the unreacted N-oxide was recovered by silica gel chromatography, which demonstrates that oxide decomposition does not occur.

TABLE 1
Regloselective Direct Arylation of Pyridine N-Oxides^{a}

| entry | N-oxide | aryl halide | product | yield^{b} |
|---|---|---|---|---|
| 1 | 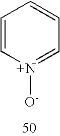 | 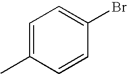 | 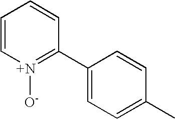 | 91 |
| 2345 | 50505050 | 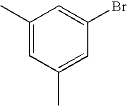 | 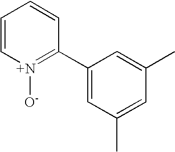 | 9589^{c}76^{d}45^{e} |
| 678 | 505050 | 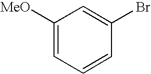 | 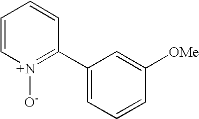 | 9793^{c}75^{d} |
| 9 | 50 | 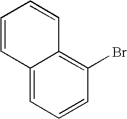 | 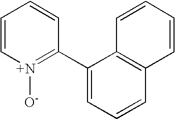 | 88 |
| 10 | 50 | 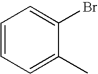 | 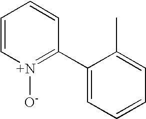 | 87 |
| 11 | 50 | 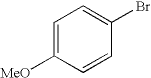 | 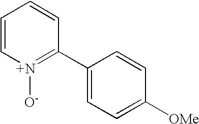 | 80 |
| 12 | 50 | 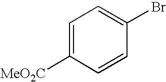 | 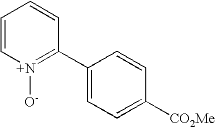 | 74 |
| 13 | 50 | 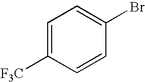 | 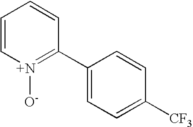 | 76 |
| 14 | 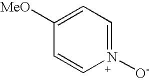 | 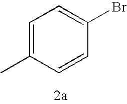 | 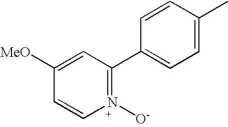 | 80 |
| 15 | 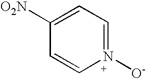 | 2a | 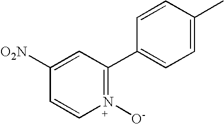 | 78 |

^{a}Conditions: aryl halide (1 equiv), pyridine N-oxide (4 equiv), K₂CO₃ (2 equiv), Pd(OAc)₂ (0.05 equiv), and PtBu₃-HBF₄ (0.15 equiv) in toluene (0.3 M) at 110° C. overnight.
^{b}Isolated yields.
^{c}With 3 equiv of 50.
^{d}With 2 equiv of 50.
^{e}With 1 equiv of 50.

Illustrative examples of the reaction scope are outlined in Table 1. Preferably, uncontrolled heating of the reaction media should be avoided, since It is known in the art that pyridine N-oxides exothermically decompose at very high temperature.¹⁷ A wide variety of compounds bearing various substituent types and at various positions can be used in the coupling process according to the invention. Both electron-rich (Table 1, entries 6-8 and 11) and electron-poor (Table 1, entries 12 and 13) aryl bromides can be used, so as more sterically encumbered ortho-substituted arenes (Table 1, entries 9 and 10). The effect of substitution on the pyridine N-oxide has also been examined. The presence of both electron-donating and -withdrawing groups is tolerated, as exemplified by the successful coupling of both 4-methoxy and 4-nitropyridine N-oxide (Table 1, entries 14 and 15). In contrast to reactions performed with many types of organometallics, these reactions are completely insensitive to the presence of water, since 5 equiv of water added at the reaction outset has no deleterious effect on the reaction outcome.

The 2-arylpyridine N-oxide products can easily be converted to the corresponding 2-aryl pyridines under mild conditions and in high yield via palladium-catalyzed reduction with ammonium formate (Table 2).^{18a} Similar yields were obtained using zinc-mediated reduction known in the art.^{18b}

TABLE 2
Deoxygenation of 2-Arylpyridine N-Oxides^{a}

| | |
|---|---|
| 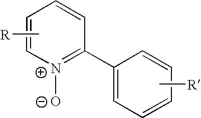 | 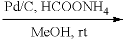 |

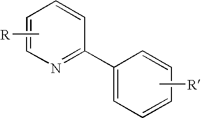

| R | R′ | yield (%) | R | R′ | yield (%) |
|---|---|---|---|---|---|
| H | 4-CH₃ | 95 | 4-OMe | 4-CH₃ | 84 |
| H | 3-OMe | 87 | H | 4-CO₂CH₃ | 87 |

^{a}Conditions: pyridine N-oxide (1 equiv), Pd/C (0.1 equiv), HCOONH₄ (10 equiv), MeOH (0.2 M), room temperature.

It can be seen that palladium-catalyzed regioselective direct arylation of pyridine N-oxides occurs in high yield with a wide range of aryl bromides. The resulting 2-arylpyridine N-oxides can be easily reduced to the free pyridine via palladium-catalyzed hydrogenolysis. Given the low cost associated with the production of pyridine N-oxides, also given the fact that pyridine N-oxides can be readily available, the coupling process according to the invention should provide a useful alternative to the problematic use of 2-pyridyl organometallics in the preparation of 2-arylpyridine N-oxides.

Recently, the potential of direct arylation as a more efficient alternative to standard cross-couplings has been recognized in the art.¹⁹ Direct arylation of N-oxides can be performed thus avoiding the use of unstable/unreactive organometallics in cross-coupling reactions.²⁰ In the context of this strategy, diazine N-oxides are more challenging than simple pyridine N-oxides since they possess a free nitrogen atom that could bind and poison the catalyst. They are also more n-electron-deficient and less nucleophilic than pyridine N-oxides. According to an aspect of the invention, conditions that enable the use of readily available, bench-stable diazine N-oxides have been established. The diazine N-oxides according to the invention are cost efficient and constitute high yielding reagents in metal-catalyzed coupling reactions.

High yielding oxidation of the corresponding free diazine could be achieved by reaction with mCPBA. The N-oxides used in this study are bench stable and show no signs of decomposition after storage in vials at room temperature for several months. To overcome catalyst poisoning associated with some N-oxide substrates, a benefical effect of metal salts including copper(I) salts was uncovered. The diazine N-oxide functionality can be easily removed after coupling or can be further converted into a wide range of other functional groups. These new reactions can be performed with aryl iodides, bromides and chlorides and include the first examples of N-oxide arylation with equimolar ratios of the two coupling partners that occur in high yield. Furthermore, the relative reactivities and regioselectivities point to C—H acidity as a critical factor in reactivity, encouraging consideration of this property in the design of other novel direct arylation processes.

Initial reaction screens with N-oxides 60, 70 and 80 under previously described conditions lead to disappointing results, probably due to the fact that the N-oxides were only sparingly soluble in toluene. The reaction conditions were reinvestigated. These efforts lead to the discovery that dioxane provides superior conversions with N-oxides 60 and 80 giving the cross coupled products 81 and 82 in 75% and 72% yields respectively (Table 3, entries 1 and 2). These two substrates actually exhibit superior reactivity compared to pyridine N-oxide as demonstrated by a competition experiment between 80 and pyridine N-oxide which results in exclusive arylation of 60 (Table 3, entry 4). In contrast to the excellent results obtained with 60 and 80, pyrimidine N-oxide 70 reacts in low yield (Table 3, entry 3).

TABLE 3
Establishment of Reaction Conditions

| Entry | N-Oxide | Aryl Halide | Additive | Product | Yield (%)^{a} |
|---|---|---|---|---|---|
| 1 | 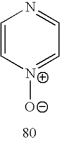 | 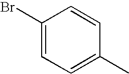 | none | 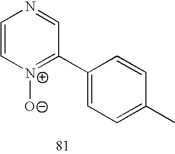 | 75 |
| 2 | 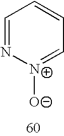 | 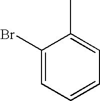 | none | 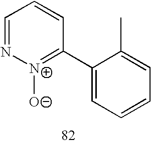 | 72 |
| 3 | 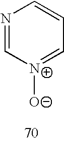 | 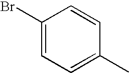 | none | 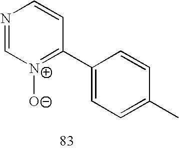 | 17 |
| 4 | 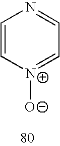 | 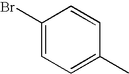 | 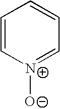 | 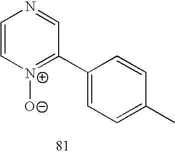 | 70 |
| 5 | 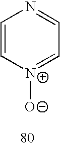 | 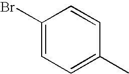 | 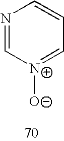 | 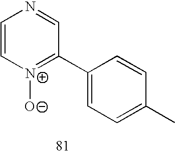 | 9 |
| 6 | 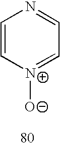 | 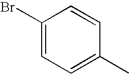 | 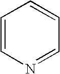 | 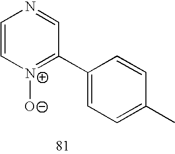 | 69 |
| 7 | 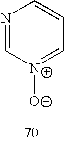 | 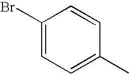 | CuCN(10 mol %) | 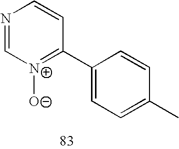 | 61^{b} |

Conditions: The N-oxide (2 equiv.), aryl halide, Pd(OAc)₂ (5 mol %), Pt-Bu₃-HBF₄ (15 mol %), K₂CO₃ (2 equiv.) and the additive (if indicated, 2 equiv.) were added to a round bottom flask followed by the addition of dioxane and heating to 110° C.
^{a}Isolated yield.
^{b}3 equiv. of 70 employed.

TABLE 4
Scope of Pyrazine N-Oxide Direct Arylation

| Entry | Equiv. 1 | ArX | Product | Yield^{a} |
|---|---|---|---|---|
| 1 | 2 | 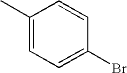 | 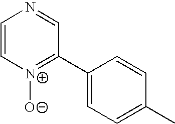 | 75 |
| 2 | 2 | 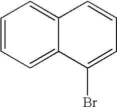 | 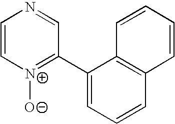 | 89 |
| 3 | 2 | 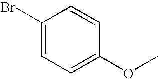 | 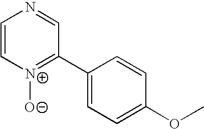 | 82 |
| 4 | 2 | 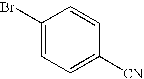 | 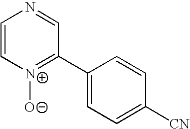 | 53 |
| 5 | 2 | 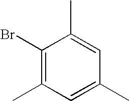 | 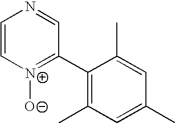 | 70 |
| 67 | 23 | 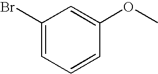 | 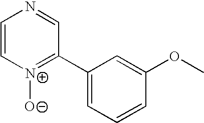 | 7284 |
| 8 | 2 | 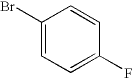 | 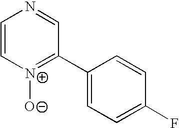 | 70 |
| 910 | 24 | 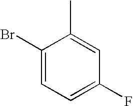 | 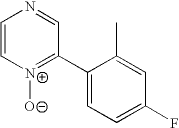 | 5096 |
| 11 | 0.3 | 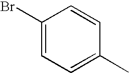 | 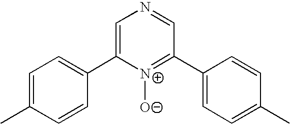 | 50 |
| 12 | 3 | 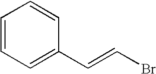 | 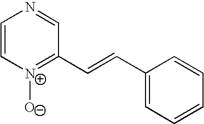 | 40 |
| 13 14 | 23 | 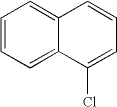 | 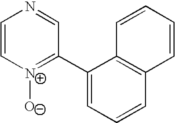 | 6068 |
| 15 | 2 | 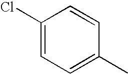 | 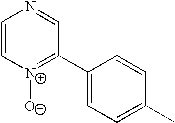 | 75 |
| 16 | 2 | 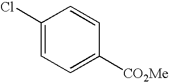 | 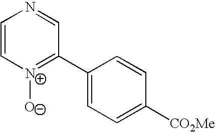 | 82 |
| 17 18 | 22^{b} | 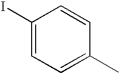 | 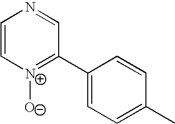 | 1777 |

Conditions: 80, aryl halide, Pd(OAc)₂ (5 mol %), Pt-Bu₃-HBF₄ (15 mol %) and K₂CO₃ (2 equiv.) were added to a round bottom flask followed by the addition of dioxane and heating to 110° C.
^{a}Isolated yield.
^{b}Ag₂CO₃ (0.5 equiv.) added.

Further investigations revealed that the poor outcomes associated with 70 are not due to low reactivity alone. For example, the addition of pyrimidine N-oxide 70 to a reaction with pyrazine N-oxide 80 results in the exclusive formation of 81 but in a significantly lower yield compared to a reaction performed in the absence of 70, 9% vs. 75% yield (Table 3, entry 5 vs. 1). Why catalyst inhibition occurs with 70 and not with 60 or 80 is a focus of ongoing study. It is noteworthy that resonance contributions for 70 induce different properties compared to those of 60 and 80. For example, distribution of the positive charge within the ring places a positive charge on the free nitrogen of 60 and 80 but not on 70. This may result in a diminished capacity to bind to palladium and explain the experimental observations. On the other hand, mesomeric resonance forms where electrons are pushed from the oxyanion into the ring put negative charges on the free nitrogen of 60 and 80 but not on 70. This should produce a trend opposite to that predicted above and to that obtain experimentally. We note that neither pyridine N-oxide nor pyridine poison the reaction of 80 (Table 3, entries 4 and 6) indicating that these deleterious effects are special to the pyrimidine N-oxide motif. Other poisoning studies where the positions adjacent to the N-oxide functional group of pyrimidine N-oxide were blocked with aryl groups also resulted in catalyst inhibition indicating that an interaction with either of these positions may not be responsible for the poor reaction of 70.

TABLE 5
Scope of Diazine N-Oxide Direct Arylation

| Entry | N-Oxide | N-Oxide Equiv. | Aryl Halide | Product | % Yield^{a} |
|---|---|---|---|---|---|
| 1 | 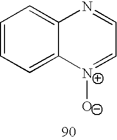 | 1 | 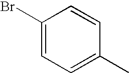 | 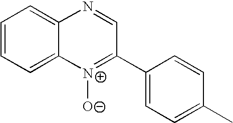 | 68 |
| 23 | 90 | 12 | 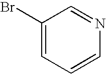 | 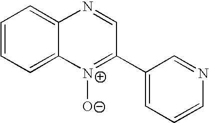 | 5080 |
| 4 | 90 | 1 | 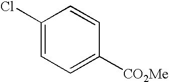 | 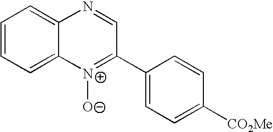 | 64 |
| 5 | 90 | 1 | 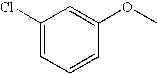 | 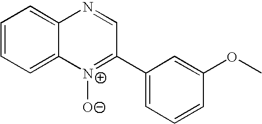 | 57 |
| 6 | 90 | 1 | 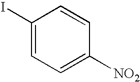 | 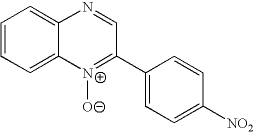 | 70^{b} |
| 7 | 90 | 1 | 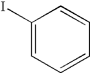 | 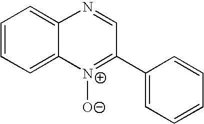 | 84^{b} |
| 89 | 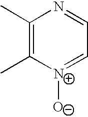 | 23 | 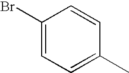 | 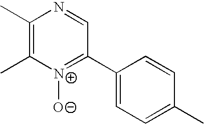 | 4856 |
| 10 11 | 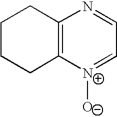 | 23 | 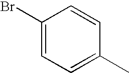 | 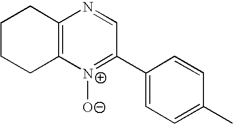 | 5256 |
| 12 | 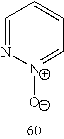 | 2 | 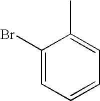 | 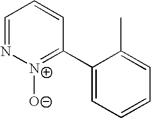 | 76 |
| 13 | 60 | 2 | 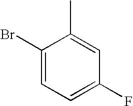 | 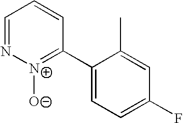 | 74 |
| 14 | 60 | 2 | 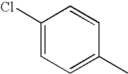 | 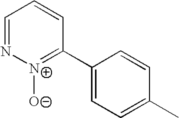 | 73 |
| 15 | 60 | 2 | 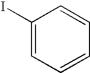 | 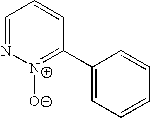 | 92^{b,c} |
| 16 | 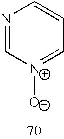 | 3(10 mol %CuCN) | 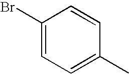 | 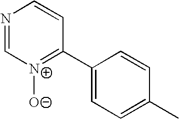 | 61 |
| 17 | 70 | 3(10 mol %CuCN) | 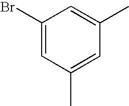 | 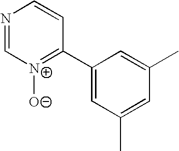 | 55 |
| 18 | 70 | 3(10 mol %CuCN) | 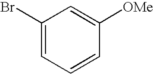 | 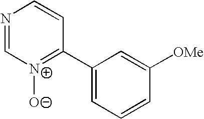 | 62 |
| 19 | 70 | 3(10 mol %CuCN) | 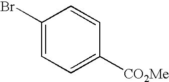 | 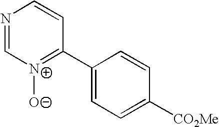 | 50 |

Conditions: Diazine N-oxide, aryl halide, Pd(OAc)₂ (5 mol %), Pt-Bu₃-HBF₄ (15 mol %) and K₂CO₃ (2 equiv.) added to a round bottom flask followed by the addition of dioxane and heating to 110° C.
^{a}Isolated yield.
^{b}Ag₂CO₃ (0.5 equiv.) added.
^{c}Performed on a 1 gram scale.

TABLE 6
N-Oxide Deoxygenation

| | | Deoxygenation | | |
|---|---|---|---|---|
| Entry | N-Oxide | Method | Product | Yield^{a} |
| 1 | 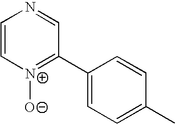 | A | 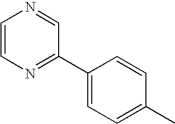 | 86 |
| 2 | 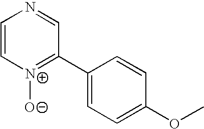 | A | 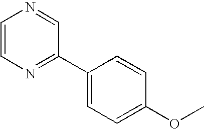 | 82 |
| 3 | 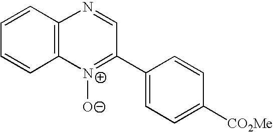 | A | 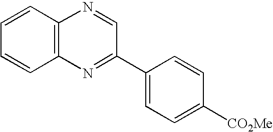 | 98 |
| 4 | 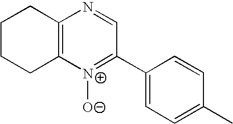 | A | 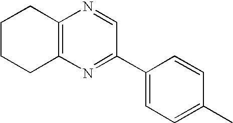 | 84 |
| 5 | 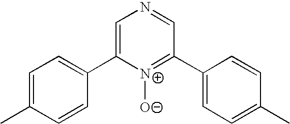 | A | 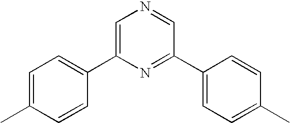 | 76 |
| 67 | 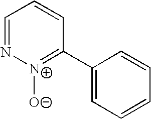 | AB | 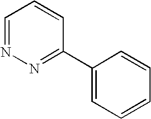 | 0^{b}87 |
| 8 | 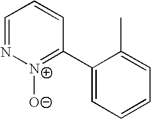 | B | 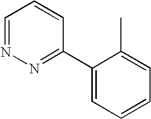 | 70 |
| 9 | 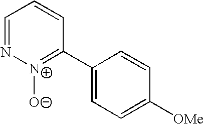 | B | 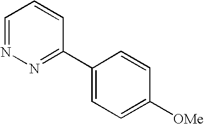 | 70 |
| 10 | 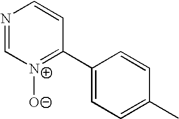 | B | 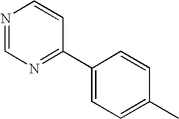 | 81 |

Conditions: Method A: Pd/C (10 mol %), NH₄HCO₂, MeOH; Method B: Pd/C, H₂, NH₄OH.
^{a}Isolated yield.

To overcome catalyst inhibition, a variety of additives were investigated including phosphines, halides and metals. Copper(I) salts such as CuCl, CuBr and CuCN were used. For reasons of ease of handling, CuCN was selected for further optimization and it was determined that the addition of 10 mol % CuCN to the new arylation conditions generates 83 in 61% isolated yield as one regioisomer. Use of CuCN may result in the in situ formation of a more nucleophilic heteroarylcopper species or reversibly bind the free nitrogen atom.

The scope of these transformations with respect to the aryl halide was evaluated with pyrazine N-oxide 80 (Table 4). High yielding arylations can be obtained not only with aryl bromides, but also with aryl iodides (Table 4, entries 17 and 18) even aryl chlorides (Table 4, entries 13 to 16). With aryl iodides, Ag₂CO₃ is optionally employed as an additive. A variety of substituents are tolerated on the aryl halide including electron-donating (Table 4, entries 3, 6 and 7) and electron-withdrawing groups (Table 4, entries 4, 8-10 and 16). More sterically encumbered aryl halides may also be employed (Table 4, entries 2, 5, 9, 10, 13 and 14). If an excess of aryl halide is used compared to the pyrazine N-oxide, the product of double direct arylation can be obtained in 50% isolated yield (Table 4, entry 11).

The scope of diazine N-oxide substrates was also evaluated (Table 5). Quinoxaline N-oxide 90 is an excellent substrate in these reactions allowing an equimolar ratio of the N-oxide and aryl halide to be used for the first time (Table 5, entries 1 to 7). More sterically encumbered alkyl substituted pyrazine N-oxides may also be reacted in synthetically useful yields (Table 5, entries 8 to 11). Different aryl halides were also examined in reactions with both pyridazine N-oxide 60 (Table 5, entries 12 to 15) and pyridimine N-oxide 70 (Table 5, entries 16 to 19). With 70, 10 mol % CuCN can be added to the reaction to help achieve the cross-coupling. In each case useful yields of the cross-coupled product are obtained.

If desired, direct arylation products can be easily deoxygenated (Table 6). With pyrazine N-oxides, treatment with ammonium formate and palladium/carbon in methanol at room temperature gives the corresponding free base in excellent yields (Table 6, method A, entries 1 to 5). This protocol may be Incompatible with pyridazine N-oxides, however (Table 6, entry 6). An extensive survey of reductive methods for N-oxide lead to the discovery that high yields can be obtained with catalytic Pd/C in ammonium hydroxide under a hydrogen atmosphere (Table 6, method B, entries 7 to 9). Pyrimidine N-oxide direct arylation products may also be deoxygenated by this second protocol in high yield (Table 6, entry 10).

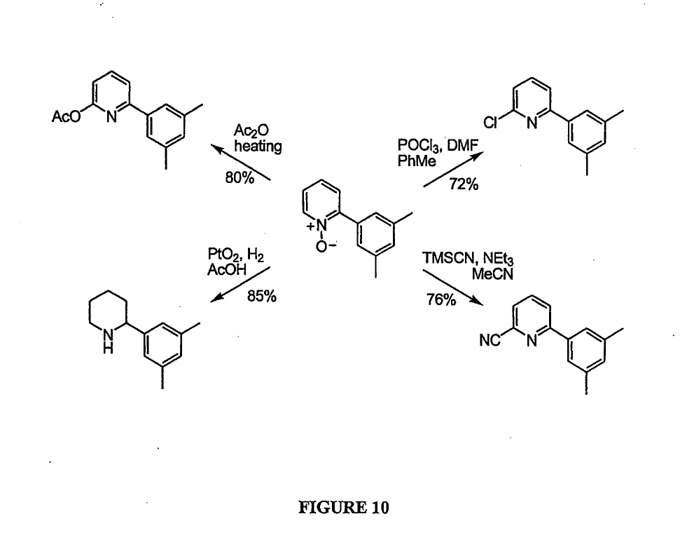

N-Oxides are key intermediates in many processes that introduce functionality adjacent to the nitrogen atom as illustrated in FIG. 10. For example, a new carbon-oxygen bond adjacent to the nitrogen atom may be formed by reaction with acetic anhydride and heating to give 101.²¹ A second direct arylation can also add a second aromatic group as in the formation of 102. Alternatively, the N-oxide may be converted to chloropyrazine 103 by reaction with POCl₃²² and subsequently used in a wide range of palladium-catalyzed cross-coupling reactions. To illustrate this possibility, a Buchwald-Hartwig amination was performed, giving 104 in 70% yield.²³ Chloride 103 may also be treated with alkoxides to give compounds such as 105 in good yield. The diazine N-oxide ring may also be reduced to arylpiperazine 106 in 68% yield by treatment with PtO₂ and H₂.

In conclusion, diazine N-oxides are convenient, inexpensive, and readily available replacements for problematic diazine organometallics in palladium-catalyzed coupling reactions. To achieve this reactivity, a variety of metal salts including copper salts may be used and the products can be further converted into a wide range of substituted nitrogen heterocycles by taking advantage of the N-oxide functionality. This chemistry should be of considerable use in the synthesis of these medicinally or industrially important compounds.

### General Methods

All experiments were carried out under an atmosphere of nitrogen. ¹H and ¹³C NMR were recorded in CDCl₃ (with Me₄Si as an internal standard) or (CD₃)₂CO or (CD₃)₂SO solutions using a Bruker AVANCE 300 or a Bruker AVANCE 400 or a Varian 500 spectrometer. High-resolution mass spectra were obtained on a Kratos Concept IIH. Infra-Red analysis was performed with a Bruker EQUINOX 55. HPLC analysis was performed on Waters apparatus using photodiode array detector. HPLC Grade THF, Et₂O, Benzene, Toluene and CH₂Cl₂ are dried and purified via MBraun SP Series solvent purification system. Triethylamine was freshly distilled from NaOH before every use. Dimethyl-acetamide was degassed with N₂ before every use. Palladium and Copper complexes were stored in a dessicator and were weighed out to air unless otherwise specified. All other reagents and solvents were used without further purification from commercial sources. Unless noted below, all other compounds have been reported in the literature or are commercially available.

### General Procedure 1: Diazine Oxidation

The appropriate diazine (1 equiv.) and mCPBA (1 equiv.) were dissolved in DCM (0.2 M). The reaction was allowed to stir for 16 hours. PPh₃ (0.5 equiv.) was then added to reduce any unreacted peracid and the mixture was stirred for an additional 4 h. The volatiles were evaporated under reduce pressure and the residue was purified via silica gel column chromatography.

### General Procedure 2: Palladium-Catalyzed Direct Arylation with Aryl Chlorides and Bromides.

To a dried flask was added the diazine N-oxide (1.0 to 3.0 equiv.), K₂CO₃ (2.0 equiv.), Pd(OAc)₂ (5 mol %) and HP(t-Bu)₃BF₄ (15 mol %). If the arylhalide is a solid, it is added at this point (1.0 equiv.). The flask and its contents were then purged under nitrogen for 10 minutes. If the aryl halide is a liquid, it is added via syringe after purging, followed by the addition of degassed dioxane (to produce a reaction concentration of 0.3 M relative to the halide). The reaction mixture was then heated at 110° C. until the reaction was complete, after which the volatiles were removed under reduced pressure and the residue was purified via silica gel column chromatography.

### General Procedure 3: Palladium-Catalyzed Direct Arylation with with Aryl Iodides.

To a dried flask was added the diazine N-oxide (1.0 to 3.0 equiv.), K₂CO₃ (2.0 equiv.), Pd(OAc)₂ (5 mol %), HP(t-Bu)₃BF₄ (15 mol %) and Ag₂CO₃ (0.5 eq.). If the arylhalide is a solid, it is added at this point (1.0 equiv.). The flask and its contents were then purged under nitrogen for 10 minutes. If the aryl halide is a liquid, it is added via syringe after purging, followed by the addition of degassed dioxane (to produce a reaction concentration of 0.3 M relative to the halide). The reaction mixture was then heated at 110° C. until the reaction was complete, after which the volatiles were removed under reduced pressure and the residue was purified via silica gel column chromatography.

### General Procedure 4: Intermolecular Palladium-Catalysed Direct Arylation with Pyrimidine N-oxides.

To a dried flask was added the diazine N-oxide (1.0 to 3.0 equiv.), K₂CO₃ (2.0 equiv.), Pd(OAc)₂ (5 mol %), HP(t-Bu)₃BF₄(15 mol %) CuCN (10 mol %). If the arylhalide is a solid, it is added at this point (1.0 equiv.). The flask and its contents were then purged under nitrogen for 10 minutes. If the aryl halide is a liquid, it is added via syringe after purging, followed by the addition of degassed dioxane (to produce a reaction concentration of 0.3 M relative to the halide). The reaction mixture was then heated at 110° C. until the reaction was complete, after which the volatiles were removed under reduced pressure and the residue was purified via silica gel column chromatography.

### General Procedure 5: Reduction of the N-Oxide Moiety (Method A)

Ammonium formate (˜10 equiv.) or H₂ was added to a stirring methanol (0.3M) solution of the N-oxide (1.0 eq.) and Pd/C (0.1 eq.) in a round bottom flask. When the reaction was deemed complete by TLC analysis, the reaction was filtered through celite and evaporated under reduced pressure. The residue was then purified via silica gel chromatography.

### General Procedure 6: Reduction of the N-Oxide Moiety (Method B)

A solution of N-oxide (1.0 eq.), Pd/C (0.1 eq.) in NH₄OH (0.2M) was reacted under an atmosphere of H₂ in a round bottom flask. When the reaction was deemed complete by TLC analysis, the reaction was filtered through celite and evaporated under reduced pressure. The residue was then purified via silica gel chromatography.

### Compound Characterization

### Oxidized Diazines

Pyrazine N-oxide (80)

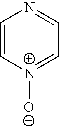

Synthesized according to general procedure 1. Purification via silica gel column chromatography using 100% EtOAc then a mixture of 20% MeOH/EtOAc gave a white solid (88%).

¹H NMR (300 MHz, CDCl₃, 293K, TMS): δ 8.50 (2H, d, J=3.9 Hz), 8.14 (2 H, d, J=4.8 Hz).

¹³C NMR (75 MHz, CDCl₃, 293K, TMS): 147.8, 134.0.

HRMS calculated for C₄H₄N₂O₁ (M+) 96.0324; Found: 96.0295.

Melting point ° C.: 103.2-104.5

IR (vₘₐₓ/cm⁻¹): 3120, 3088, 1595, 861, 847, 838.

Rf (20% MeOH/EtOAc): 0.3

Quinoxaline N-oxide (90)

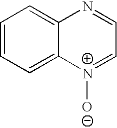

Synthesized according to general procedure 1. Purification via silica gel column chromatography using 100% EtOAc gave a yellow solid (70%). Spectral data is identical to previous reports.²⁴

### 2,3-dimethylpyrazine N-oxide (Table 5, entries 8 and 9)

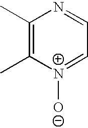

Synthesized according to general procedure 1. Purification via silica gel column chromatography using 100% EtOAc then a mixture of 10% MeOH/EtOAc gave a white solid (88%). Spectral data is identical to previous reports²⁵.

### 5,6,7,8-tetrahydroquinoxaline N-oxide (Table 5, entries 10 and 11)

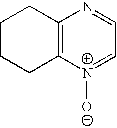

Synthesized according to general procedure 1. Purification via silica gel column chromatography using 100% EtOAc then a mixture of 5% MeOH/EtOAc gave a white solid (77%).

¹H NMR (500 MHz, CDCl₃, 293K, TMS): δ 8.26 (1H, d, J=3.5 Hz), 8.03 (1H, d, J=4 Hz), 2.93 (4H, dt, J=6 and 19 Hz), 1.93-1.89 (4H, m).

¹³C NMR (125 MHz, CDCl₃, 293K, TMS): 157.3, 143.5, 143.2, 131.2, 31.7, 23.5, 21.6, 21.2.

HRMS calculated for C₈H₁₀N₂O₁ (M+) 150.0793; Found: 150.0789.

Melting point ° C.: 74.1-75.0

IR (vₘₐₓ/cm⁻¹): 3114, 2939, 2879, 1584, 1453, 1296, 975, 830.

Rf (5% MeOH/EtOAc): 0.4

### Pyridazine N-oxide (60)

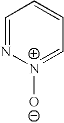

Synthesized according to general procedure 1. Purification via silica gel column chromatography using 100% EtOAc then a mixture of 20% MeOH/EtOAc gave a brownish oil (quant.).

¹H NMR (300 MHz, CDCl₃, 293K, TMS): δ 8.59 (1H, s), 8.33 (1H, d, J=6.6 Hz), 7.92-7.87 (1H, m), 7.29 (1H, dd, J=5.7 and 6.6 Hz).

¹³C NMR (75 MHz, CDCl₃, 293K, TMS): 149.8, 133.9, 133.6, 115.9.

HRMS calculated for C₄H₄N₂O₁ (M+) 96.0324; Found: 96.0318.

IR (vₘₐₓ/cm⁻¹): 3109, 1583, 1416, 982, 847.

Rf (20% MeOH/EtOAc): 0.3

### Pyrimidine N-oxide (70)

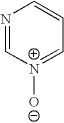

Synthesized according to general procedure 1. Purification via silica gel column chromatography using 100% EtOAc then a mixture of 15% MeOH/EtOAc gave a white solid (92%).

¹H NMR (300 MHz, CDCl₃, 293K, TMS): δ 9.03 (1H, s), 8.47 (1H, d, J=6.6 Hz), 8.30 (¹H, d, J=4.2 Hz), 7.39 (1H, t, J=5.4 Hz).

¹³C NMR (75 MHz, CDCl₃, 293K, TMS): 149.6, 144.1, 143.5, 121.0.

HRMS calculated for C₄H₄N₂O₁ (M+) 96.0324; Found: 96.0304.

Melting point ° C.: 92.0-92.5

IR (vₘₐₓ/cm⁻¹): 3083, 1653, 1541, 1414, 1251, 843.

Rf (15% MeOH/EtOAc): 0.3

### 2-Phenylpyrimidine

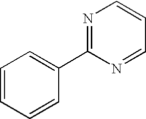

To a dried flask was added the 2-chloropyrimidine (0.50 g, 4.37 mmol), phenyl-boronic acid (0.69 g, 5.68 mmol), Na₂CO₃ (0.92 g, 8.70 mmol), PdCl₂ (38.7 mg, 0.22 mmol) and dppb (92.9 mg, 0.22 mmol). The mixture was then purged under nitrogen for 10 minutes, followed by the addition of a degassed mixture of toluene (12 mL), water (6 mL), ethanol (2 mL). The reaction mixture was allowed to stir at 100° C. After 20 h, the mixture was filtered on a celite pad, then the volatiles were removed under reduced pressure. Purification via silica gel column chromatography using a mixture of 10% Et₂O/DCM gave a white solid (65%). Spectral data is identical to previous reports.²⁶

### 2-Phenylpyrimidine N-oxide

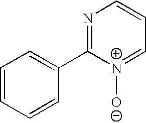

Synthesized according to general procedure 1. Purification via silica gel column chromatography using 100% EtOAc then a mixture of 10% MeOH/EtOAc gave a beige solide.

¹H NMR (300 MHz, CDCl₃, 293K, TMS): δ 8.50-8.45 (3H, m), 8.32 (1H, dd, J=1.2 and 3.0 Hz), 7.51-7.49 (3H, m), 7.18 (1H, dd, J=3.0 and 4.5 Hz).

¹³C NMR (75 MHz, CDCl₃, 293K, TMS): 156.4, 146.6, 143.5, 131.3, 131.1, 129.7, 127.9, 119.2.

HRMS calculated for C₁₀H₈N₂O (M+) 172.0637; Found: 172.0647.

Melting point ° C.: 89.7-91.2.

IR (vₘₐₓ/cm⁻¹): 3097, 2933, 1534, 1400, 1249, 722.

Rf (10% MeOH/EtOAc): 0.1

### 2-Arylpyrazine N-Oxides

### 2-p-Tolylpyrazine N-oxide (81)

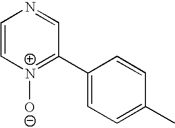

Synthesized according to general procedure 2 employing the corresponding aryl bromide and chloride or 60 with the corresponding aryl iodide. Purification via silica gel column chromatography using 100% DCM then a mixture of 20% Acetone/DCM gave a white solid, 72% (from the bromide), 75% (from the chloride) and 77% (from the iodide).

¹H NMR (300 MHz, CDCl₃, 293K, TMS): δ 8.63 (1H, s), 8.37 (1H, s), 8.20 (1H, s), 7.72 (2H, d, J=8.1 Hz), 7.33 (2H, d, J=7.8 Hz), 2.43 (3H, s).

¹³C NMR (75 MHz, CDCl₃, 293K, TMS): 148.2, 145.2, 144.6, 140.8, 134.2, 129.8, 129.0, 125.9, 21.5.

HRMS calculated for C₁₁H₁₀N₂O₁ (M+) 186.0793; Found: 186.0790.

Melting point ° C.: 136.1-137.0

IR (vₘₐₓ/cm⁻¹): 3110, 3038, 2925, 2850, 1590, 1301, 869, 821.

Rf (10% Acetone/DCM): 0.25

### 2-(Naphthalen-1-yl)pyrazine N-oxide (Table 4, entries 2, 13 and 14)

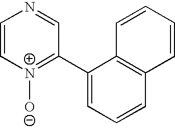

Synthesized according to general procedure 2 employing the corresponding aryl bromide and chloride. Purification via silica gel column chromatography using 100% DCM then a mixture of 10% Acetone/DCM gave a brown oil, 89% from the bromide and 60% from the chloride.

¹H NMR (300 MHz, CDCl₃, 293K, TMS): δ 8.61 (1H, s), 8.48 (1H, d, J=3.9 Hz), 8.26 (1H, d, J=4.2 Hz), 8.00 (1H, d, J=7.8 Hz), 7.93-7.87 (1H, m), 7.59-7.37 (5H, m).

¹³C NMR (75 MHz, CDCl₃, 293K, TMS): 150.1, 147.1, 145.7, 134.8, 133.9, 131.5, 131.3, 129.2, 128.9, 127.6, 127.0, 125.7, 125.3.

HRMS calculated for C₁₄H₁₀N₂O₁ (M+) 222.0793; Found: 222.0775.

IR (vₘₐₓ/cm⁻¹): 3057, 3010, 2923, 2853, 1578, 1301, 873, 801, 776.

Rf (10% Acetone/DCM): 0.5

### 2-(4-Methoxyphenyl)pyrazine N-oxide (Table 4, Entry 3)

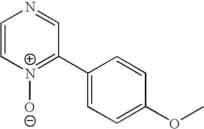

Synthesized according to general procedure 2. Purification via silica gel column chromatography using 100% DCM then a mixture of 15% Acetone/DCM gave a beige solid (82%).

¹H NMR (300 MHz, CDCl₃, 293K, TMS): δ 8.62 (1H, s), 8.32 (1H, s), 8.18 (1H, s), 7.82 (2H, d, J=8.4 Hz), 7.04 (2H, d, J=8.7 Hz), 3.87 (3H, s).

¹³C NMR (75 MHz, CDCl₃, 293K, TMS): 161.0, 147.9, 144.7, 144.1, 134.3, 130.6, 120.9, 113.9, 55.3.

HRMS calculated for C₁₁H₁₀N₂O₂ (M+) 202.0742; Found: 202.0755.

Melting point ° C.: 145.0-146.2

IR (vₘₐₓ/cm⁻¹): 3164, 3082, 2965, 2840, 1456, 1294, 861, 838, 820, 803.

Rf (10% Acetone/DCM): 0.2

### 4-(Pyrazin-2-yl)benzonitrile N-oxide (Table 4, Entry 4)

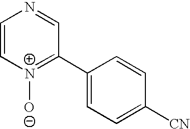

Synthesized according to general procedure 2. Purification via silica gel column chromatography using 100% DCM then a mixture of 15% Acetone/DCM gave a white solid (53%).

¹H NMR (300 MHz, CDCl₃, 293K, TMS): δ 8.66 (1H, s), 8.48 (1H, d, J=3.9 Hz), 8.24 (1H, d, J=4.2 Hz), 7.97 (2H, d, J=8.1 Hz), 7.82 (2H, d, J=8.1 Hz).

¹³C NMR (75 MHz, CDCl₃, 293K, TMS): 148.1, 146.6, 142.8, 134.5, 133.2, 132.2, 129.8, 118.0, 113.9.

HRMS calculated for C₁₁H₇N₃O₁ (M+) 197.0589; Found: 197.0565.

Melting point ° C.: 194.7-196.5.

IR (vₘₐₓ/cm⁻¹): 3098, 3067, 3046, 2240, 1589, 1389, 870, 836.

Rf (15% Acetone/DCM): 0.3

### 2-Mesitylpyrazine N-oxide (Table 4, Entry 5)

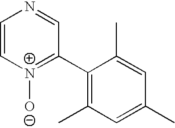

Synthesized according to general procedure 2. Purification via silica gel column chromatography using 100% DCM then a mixture of 15% Acetone/DCM gave a beige solid 70% yield with 2 eq. of the N-oxide and 76% yield with 3 eq. of the N-oxide.

¹H NMR (300 MHz, CDCl₃, 293K, TMS): δ 8.45 (1H, d, J=3.9 Hz) 8.42 (1H, s), 8.26 (1H, d, J=4.2 Hz), 7.00 (2H, s), 2.34 (3H, s), 2.07 (6H, s).

¹³C NMR (75 MHz, CDCl₃, 293K, TMS): 149.4, 146.2, 145.3, 140.0, 137.2, 134.3, 128.5, 125.7, 21.1, 19.4.

HRMS calculated for C₁₃H₁₄N₂O₁ (M+) 214.1106; Found: 214.1091.

Melting point ° C.: 118.0-119.3.

IR (vₘₐₓ/cm⁻¹): 3106, 2971, 2913, 2855, 1582, 1389, 1007, 862, 843.

Rf (10% Acetone/DCM): 0.3

### 2-(3-Methoxyphenyl)pyrazine N-oxide (Table 4, Entries 6 and 7)

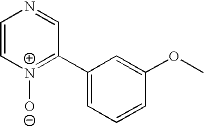

Synthesized according to general procedure 2. Purification via silica gel column chromatography using 100% DCM then a mixture of 15% Acetone/DCM gave a white solid, 72% with 2 eq. of the N-oxide and 84% yield with 3 eq. of the N-oxide).

¹H NMR (300 MHz, CDCl₃, 293K, TMS): δ 8.63 (1H, s), 8.38 (1H, d, J=3.9 Hz), 8.20 (1H, d, J=4.2 Hz), 7.46-7.29 (3H, m), 7.05 (1H, dd, J=1.8 and 8.4 Hz), 3.85 (3H, s).

¹³C NMR (75 MHz, CDCl₃, 293K, TMS): 159.4, 148.3, 145.5, 144.3, 134.4, 130.0, 129.6, 121.3, 116.2, 114.4, 55.3.

HRMS calculated for C₁₁H₁₀N₂O₂ (M+) 202.0742; Found: 202.0770.

Melting point ° C.: 89.6-90.4.

IR (vₘₐₓ/cm⁻¹): 3117, 3011, 2976, 2930, 2843, 1591, 1302, 886, 858, 848.

Rf (15% Acetone/DCM): 0.2

### 2-(4-Fluorophenyl)pyrazine N-oxide (Table 4, Entry 8)

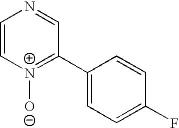

Synthesized according to general procedure 2. Purification via silica gel column chromatography using 100% DCM then a mixture of 10% Acetone/DCM, then a mixture of 15% Acetone/DCM gave a white solid (70%).

¹H NMR (400 MHz, CDCl₃, 293K, TMS): δ 8.62 (1H, s), 8.39 (1H, d, J=3.0 Hz), 8.21 (1H, d, J=3.0 Hz), 7.84 (2H, dd, J=4.2 and 6.0 Hz), 7.22 (2H, t, J=6.3 Hz).

¹³C NMR (100 MHz, CDCl₃, 293K, TMS): 163.7 (d, J=250.1 Hz), 148.1, 145.6, 143.6, 134.4, 131.3 (d, J=8.6 Hz), 124.9 (d, J=3.5 Hz), 115.8 (d, 21.8 Hz).

HRMS calculated for C₁₀H₇N₂OF (M+) 190.0542; Found: 190.0531.

Melting point ° C.: 169.5-170.1.

IR (vₘₐₓ/cm⁻¹): 3109, 3073, 3017, 1584, 1458, 1297, 832.

Rf (10% Acetone/DCM): 0.3

### 2-(4-Fluoro-2-methylphenyl)pyrazine N-oxide (Table 4, Entries 9 and 10)

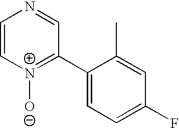

Synthesized according to general procedure 2. Purification via silica gel column chromatography using 100% DCM then a mixture of 5% Acetone/DCM, then a mixture of 10% Acetone/DCM gave a white solid, 50% yield with 2 eq. of the N-oxide and 96% yield with 4 eq. of the N-oxide.

¹H NMR (400 MHz, CDCl₃, 293K, TMS): δ 8.49 (1H, s), 8.46 (1H, d, J=4.0 Hz), 8.22 (1H, d, J=4.0 Hz), 7.27-7.22 (1H, m), 7.08-7.00 (2H, m), 2.23 (3H, s).

¹³C NMR (100 MHz, CDCl₃, 293K, TMS): 163.7 (d, J=248.3 Hz), 149.0, 146.4, 145.4, 141.5 (d, J=8.4 Hz), 134.1, 131.6 (d, J=9.0 Hz), 125.0 (d, J=3.2 Hz), 117.3 (d, J=21.6 Hz), 113.1 (d, J=21.8 Hz), 19.5 (d, J=1.4 Hz)

HRMS calculated for C₁₁H₉N₂OF (M+) 204.0699; Found: 204.0755.

Melting point ° C.: 75.0-75.4

IR (vₘₐₓ/cm⁻¹): 3083, 3025, 2925, 1582, 1455, 1297, 866.

Rf (10% Acetone/DCM): 0.45

### 2,6-dip-tolylpyrazine N-oxide (Table 2, Entry 11)

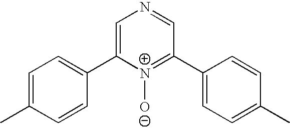

Synthesized according to general procedure 2 and using 0.3 eq. of pyrazine N-oxide. Purification via silica gel column chromatography using 100% DCM gave a beige solid (50%).

¹H NMR (300 MHz, CDCl₃, 293K, TMS): δ 8.53 (2H, s), 7.74 (4H, d, J=8.1 Hz), 7.32 (4H, d, J=7.8 Hz), 2.43 (6H, s).

¹³C NMR (75 MHz, CDCl₃, 293K, TMS): 145.9, 144.6, 140.4, 129.3, 129.1, 126.5, 21.4.

HRMS calculated for C₁₈H₁₆N₂O (M+) 276.1263; Found: 276.1279.

Melting point ° C.: 146.0-147.6

IR (vₘₐₓ/cm⁻¹): 3026, 2922, 2862, 1500, 1297, 865, 826.

Rf (100% DCM): 0.7

### 2-Styrylpyrazine N-oxide (Table 4, Entry 12)

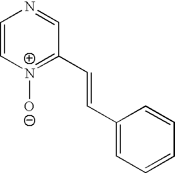

Synthesized according to general procedure 2. Purification via silica gel column chromatography using 100% DCM, then a mixture of 10% Acetone/DCM gave a brownish solid, 32% yield with 2 eq. of the N-oxide and 40% yield with 3 eq. of the N-oxide).

¹H NMR (300 MHz, CDCl₃, 293K, TMS): δ 8.82 (1H, s), 8.31-8.22 (1H, m), 8.14-8.11 (1H, m), 7.72 (1H, d, J=16.5 Hz), 7.62 (2H, dd, J=3.0 and 7.8 Hz), 7.53 (1H, d, J=16.5 Hz), 7.45-7.34 (3H, m)

¹³C NMR (75 MHz, CDCl₃, 293K, TMS): 145.8, 143.9, 136.7, 135.7, 133.9, 129.5, 128.9, 128.4, 127.5, 115.6.

HRMS calculated for C₁₂H₁₀N₂O (M+) 198.0793; Found: 198.0786.

Melting point ° C.: 152.0-153.3

IR (vₘₐₓ/cm⁻¹): 3112, 3061, 3024, 1589, 1410, 1273, 981.

Rf (10% Acetone/DCM): 0.35

### Methyl 4-(pyrazin-2-yl)benzoate N-oxide (Table 4, Entry 16)

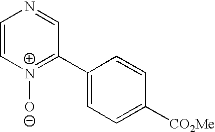

Synthesized according to general procedure 2. Purification via silica gel column chromatography using 100% DCM then a mixture of 10% Acetone/DCM gave a beige solid (82%).

¹H NMR (300 MHz, DMSO, 383K): δ 8.77 (1H, s), 8.50 (1H, d, J=4.2 Hz), 8.38 (1H, d, J=4.5 Hz), 8.08 (2H, d, J=8.7 Hz), 8.01 (2H, d, J=8.7 Hz), 3.92 (3H, s).

¹³C NMR (75 MHz, DMSO, 383K): 165.1, 147.5, 146.0, 142.0, 133.9, 133.2, 130.4, 128.8, 128.2, 51.4.

HRMS calculated for C₁₂H₁₀N₂O₃ (M+) 230.0691; Found: 230.0686.

Melting point ° C.: 215.9-217.1.

IR (vₘₐₓ/cm⁻¹): 3074, 2917, 2854, 1722, 1384, 1278, 856.

Rf (10% Acetone/DCM): 0.2

### Substituted Arylpyrazine N-Oxides

### 2-p-Tolylquinoxaline N-oxide (Table 5, Entry 1)

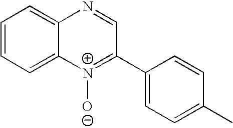

Synthesized according to general procedure 2. Purification via silica gel column chromatography using 100% DCM then a mixture of 5% Acetone/DCM gave a yellow solid (68%).

¹H NMR (300 MHz, CDCl₃, 293K, TMS): δ 8.89 (1H, s), 8.69-8.65 (1H, m), 8.13-8.10 (1H, m), 7.91 (2H, d, J=8.1 Hz), 7.81-7.73 (2H, m), 7.37 (2H, d, J=8.1 Hz), 2.44 (3H, s).

¹³C NMR (75 MHz, CDCl₃, 293K, TMS): 147.3, 144.2, 140.6, 139.2, 137.3, 130.9, 130.3, 129.8, 129.3, 129.2, 126.9, 119.2, 21.5.

HRMS calculated for C₁₅H₁₂N₂O₁ (M+) 236.0871; Found: 236.0958.

Melting point ° C.: 149.1-150.5

IR (vₘₐₓ/cm⁻¹): 3127, 3034, 2920, 2856, 1578, 1488, 1351, 818, 763, 749.

Rf (5% Acetone/DCM): 0.4

### 2-(Pyridin-3-yl)quinoxaline N-oxide (Table 5, Entries 2 and 3)

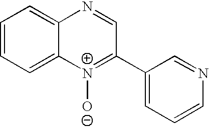

Synthesized according to general procedure 2. Purification via silica gel column chromatography using 100% DCM, then a mixture of 10% Acetone/DCM, then a mixture of 40% Acetone/DCM gave a white solid 50% with 1 eq. of the N-oxide and 80% yield with 2 eq. of the N-oxide).

¹H NMR (300 MHz, DMSO, 293K): δ 9.18 (2H, d, J=16.8 Hz), 8.72 (1H, s), 8.55-8.47 (2H, m), 8.17 (1H, d, J=7.8 Hz), 8.00-7.78 (2H, m), 7.62 (1H, m).

¹³C NMR (75 MHz, DMSO, 293K): 150.5, 149.9, 147.6, 144.2, 137.1, 136.4, 136.4, 131.7, 130.7, 129.7, 126.2, 123.2, 118.6.

HRMS calculated for C₁₃H₉N₃O₁ (M+) 223.0746; Found: 223.0726.

Melting point ° C.: 181.9-183.0

IR (vₘₐₓ/cm⁻¹): 3103, 3063, 3025, 2920, 1491, 1327, 902, 782, 770, 753.

Rf (10% Acetone/DCM): 0.1

### Methyl 4-(quinoxalin-2-yl)benzoate N-oxide (Table 5, Entry 4)

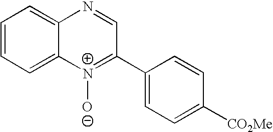

Synthesized according to general procedure 2. Purification via silica gel column chromatography using 100% DCM, then a mixture of 2.5% Acetone/DCM, then a mixture of 5% Acetone/DCM gave a beige solid (84%).

¹H NMR (300 MHz, CDCl₃, 293K, TMS): δ 8.92 (1H, s), 8.68 (1H, dd, J=1.5 and 9 Hz), 8.23 (2H, d, J=8.7 Hz), 8.16 (1H, d, J=7.8 Hz), 8.08 (2H, d, J=8.7 Hz), 7.82 (2 H, m), 3.98 (3H, s).

¹³C NMR (75 MHz, CDCl₃, 293K): 166.3, 147.0, 144.7, 138.4, 137.4, 134.2, 131.5, 131.4, 130.7, 130.1, 129.7, 129.4, 119.3, 52.4.

HRMS calculated for C₁₆H₁₂N₂O₃ (M+) 280.0848; Found: 280.0824.

Melting point ° C.: 219.3-220.0

IR (vₘₐₓ/cm⁻¹): 3116, 3061, 2987, 1715, 1491, 1349, 901, 766.

Rf (5% Acetone/DCM): 0.35

### 2-(3-Methoxyphenyl)quinoxaline N-oxide (Table 5, Entry 5)

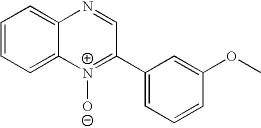

Synthesized according to general procedure 2. Purification via silica gel column chromatography using 100% DCM, then a mixture of 2% Acetone/DCM gave a yellow solid (57%).

¹H NMR (300 MHz, CDCl₃, 293K, TMS): δ 8.90 (1H, s), 8.67 (1H, d, J=7.8 Hz), 8.12 (1H, d, J=7.5 Hz), 7.83-7.74 (2H, m), 7.61 (1H, s), 7.48 (1H, m), 7.07 (1H, d, J=6.3 Hz), 3.88 (3H, s).

¹³C NMR (75 MHz, CDCl₃, 293K, TMS): 159.4, 147.3, 144.3, 139.0, 137.3, 131.1, 131.0, 130.3, 129.9, 129.6, 121.6, 119.2, 116.3, 114.5, 55.3.

HRMS calculated for C₁₅H₁₂N₂O₂ (M+) 252.0899; Found: 252.0911.

Melting point ° C.: 132.0-133.6

IR (vₘₐₓ/cm⁻¹): 3066, 3013, 2970, 2930, 1491, 1354, 1033, 760, 755.

Rf (2% Acetone/DCM): 0.3

### 2-(4-Nitrophenyl)quinoxaline N-oxide (Table 5, Entry 6)

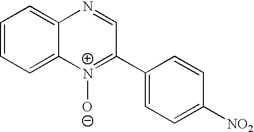

Synthesized according to general procedure 3. Purification via silica gel column chromatography using 100% DCM, then a mixture of 3% Acetone/DCM, then a mixture of 5% Acetone/DCM gave a yellow solid (70%).

¹H NMR (300 MHz, DMSO, 368 K): δ 9.09 (1H, s), 8.58 (1H, d, J=8.7 Hz), 8.42-8.28 (4H, m), 8.18 (1H, d, J=9 Hz), 8.02-7.84 (2H, m).

¹³C NMR (75 MHz, DMSO, 368K): 149.1, 148.3, 145.5, 138.0, 137.8, 137.2, 132.8, 131.8, 131.6, 130.7, 124.1, 119.7.

HRMS calculated for C₁₄H₉N₃O₃ (M+) 267.0644; Found: 267.0645.

Melting point ° C.: 255 (decomp.)

IR (vₘₐₓ/cm⁻¹): 3108, 2955, 2921, 1519, 1338, 842, 763.

Rf (5% Acetone/DCM): 0.4

### 2-Phenylquinoxaline N-oxide (Table 5, Entry 7)

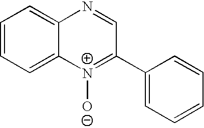

Synthesized according to general procedure 3. Purification via silica gel column chromatography using 100% DCM, then a mixture of 3% Acetone/DCM gave a beige solid (84%).

¹H NMR (300 MHz, CDCl₃, 293K, TMS): δ 8.90 (1H, s), 8.69 (1H, d, J=7.8 Hz), 8.13 (1H, d, J=9.3 Hz), 7.99 (2H, d, J=7.8 Hz), 7.79 (2H, m), 7.62-7.48 (3H, m).

¹³C NMR (75 MHz, CDCl₃, 293K, TMS): 147.4, 144.4, 139.2, 137.3, 131.1, 130.4, 130.2, 129.9, 129.8, 129.3, 128.6, 119.3.

HRMS calculated for C₁₄H₁₀N₂O (M+) 222.0793; Found: 222.0791.

Melting point ° C.: 153.4-155.0.

IR (vₘₐₓ/cm⁻¹): 3049, 3006, 1485, 1317, 893, 766.

Rf (3% Acetone/DCM): 0.3

### 2,3-dimethyl-5-p-tolylpyrazine N-oxide (Table 3, Entries 8 and 9)

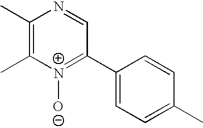

Synthesized according to general procedure 2. Purification via silica gel column chromatography using 100% DCM then a mixture of 10% Acetone/DCM gave a white solid, 40% yield with 0.5 eq. of the N-oxide, 18% yield with 1 eq. of the N-oxide, 48% yield with 2 eq. of the N-oxide and 56% with 3 eq. of the N-oxide.

¹H NMR (300 MHz, CDCl₃, 293K, TMS): δ 8.36 (1H, s), 7.67 (1H, d, J=3.9 Hz), 7.30 (1H, d, J=4.2 Hz), 2.62 (3H, s), 2.54 (3H, s), 2.42 (3H, s).

¹³C NMR (75 MHz, CDCl₃, 293K, TMS): 153.5, 143.3, 142.7, 141.8, 139.9, 129.0, 129.0, 127.0, 22.5, 21.4, 13.3.

HRMS calculated for C₁₃H₁₄N₂O₁ (M+) 214.1106; Found: 214.1117.

Melting point ° C.: 135.1-136.8.

IR (vₘₐₓ/cm⁻¹): 3028, 2996, 2918, 1585, 1464, 1300, 879, 817.

Rf (10% Acetone/DCM): 0.3

### 5,6,7,8-tetrahydro-2-p-tolylquinoxaline N-oxide (Table 5, Entries 10 and 11)

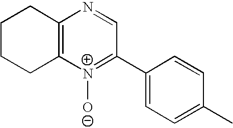

Synthesized according to general procedure 2. Purification via silica gel column chromatography using 100% DCM, then a mixture of 5% Acetone/DCM gave a white solid, 34% yield with 1 eq. of the N-oxide, 52% yield with 2 eq. of the N-oxide and 56% yield with 3 eq. of the N-oxide.

¹H NMR (300 MHz, CDCl₃, 293K, TMS): δ 8.41 (1H, s), 7.68 (2H, d, J=8.1 Hz), 7.30 (2H, d, J=8.1 Hz), 3.03-2.90 (4 H. m), 2.01-1.84 (4H, m).

¹³C NMR (75 MHz, CDCl₃, 293K, TMS): 154.8, 143.7, 143.4, 141.7, 139.9, 129.1, 126.9, 31.8, 24.1, 21.7, 21.5, 21.4.

HRMS calculated for C₁₅H₁₆N₂O₁ (M+) 240.1263; Found: 240.9852.

Melting point ° C.: 149.0-151.6.

IR (vₘₐₓ/cm⁻¹): 3031, 2950, 2871, 1584, 1459, 1300, 819.

Rf (10% Acetone/DCM): 0.45

### 2-Arylpyridazine N-Oxides

### 3-o-Tolylpyridazine N-oxide (82)

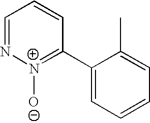

Synthesized according to general procedure 2. Purification via silica gel column chromatography using 100% DCM then a mixture of 10% Acetone/DCM gave a brownish oil (72%).

¹H NMR (300 MHz, CDCl₃, 293K, TMS): δ 8.48 (1H, d, J=7.2 Hz), 7.63 (1H, dd, J=2.4 and 6 Hz), 7.41-7.20 (4H, m), 7.13 (1H, dd, J=5.4 and 6 Hz), 2.23 (3H, s).

¹³C NMR (75 MHz, CDCl₃, 293K, TMS): 149.6, 145.7, 137.6, 135.4, 131.6, 130.2, 129.8, 129.1, 125.9, 115.7, 19.2.

HRMS calculated for C₁₁H₁₀N₂O₁ (M+) 186.0793; Found: 186.0790.

IR (vₘₐₓ/cm⁻¹): 3058, 2955, 2866, 1539, 1369, 768.

Rf (10% Acetone/DCM): 0.35

### 3-(4-Fluoro-2-methylphenyl)pyridazine N-oxide (Table 5, Entry 13)

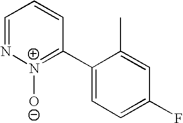

Synthesized according to general procedure 2. Purification via silica gel column chromatography using 100% DCM then a mixture of 5% Acetone/DCM, then a mixture of 15% Acetone/DCM gave a brownish oil (74%).

¹H NMR (300 MHz, CDCl₃, 293K, TMS): δ 8.54-8.44 (1H, m), 7.64 (1H, dd, J=2.4 and 9.0 Hz), 7.23-7.13 (2H, m), 7.04-6.95 (2H, m), 2.23 (3H, s).

¹³C NMR (75 MHz, CDCl₃, 293K, TMS): 163.3 (d, J=247.7 Hz), 149.8, 144.9, 140.6 (d, J=8.5 Hz), 135.5, 131.0 (d, J=9 Hz), 127.6 (d, J=3.2 Hz), 117.2 (d, J=21.6 Hz), 115.7, 113.0 (d, 21.8 Hz), 19.4.

HRMS calculated for C₁₁H₉N₂OF (M+) 204.0699; Found: 204.0717.

IR (vₘₐₓ/cm⁻¹): 3073, 3033, 2932, 1572, 1449, 1303, 871.

Rf (10% Acetone/DCM): 0.25

### 3-p-Tolylpyridazine N-oxide (Table 5, Entry 14)

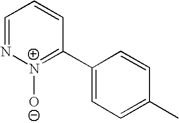

Synthesized according to general procedure 2. Purification via silica gel column chromatography using 100% DCM then a mixture of 5% Acetone/DCM gave white solid (73%).

¹H NMR (300 MHz, CDCl₃, 293K, TMS): δ 8.40 (1H, m), 7.75 (1H, dd, J=2.1 and 6.0 Hz), 7.71 (2H, d, J=8.1 Hz), 7.28 (2H, d, J=7.8 Hz), 7.13 (1H, dd, J=5.1 and 6.0 Hz), 2.40 (3H, s).

¹³C NMR (75 MHz, CDCl₃, 293K, TMS): 148.7, 144.3, 140.3, 134.4, 129.0, 128.7, 128.3, 116.3, 21.3.

HRMS calculated for C₁₁H₁₀N₂O (M+) 186.0793; Found: 186.0768.

Melting point ° C.: 160.1-161.6.

IR (vₘₐₓ/cm⁻¹): 3031, 2918, 1451, 1359, 1291, 828, 783.

Rf (10% Acetone/DCM): 0.4

### 3-Phenylpyridazine N-oxide (Table 5, Entry 15)

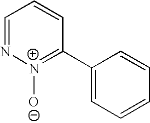

Synthesized according to general procedure 3. Purification via silica gel column chromatography using 100% DCM then a mixture of 10% Acetone/DCM gave a white solid (91%).

¹H NMR (300 MHz, CDCl₃, 293K, TMS): δ 8.47-8.38 (1H, m), 7.86-7.74 (3H, m), 7.50-7.43 (3H, m), 7.15 (1H, dd, J=5.1 and 9 Hz).

¹³C NMR (75 MHz, CDCl₃, 293K, TMS): 149.1, 144.3, 134.6, 131.3, 130.0, 128.8, 128.4, 116.3.

HRMS calculated for C₁₀H₈N₂O (M+) 172.0637; Found: 172.0612.

Melting point ° C.: 124.3-126.1.

IR (vₘₐₓ/cm⁻¹): 3078, 2920, 1542, 1375, 871, 685.

Rf (10% Acetone/DCM): 0.3

### 2-Arylpyrimidine N-Oxides

### 4-p-Tolylpyrimidine N-oxide (Table 5, Entry 16)

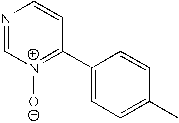

Synthesized according to general procedure 4. Purification via silica gel column chromatography using 100% DCM then a mixture of 15% Acetone/DCM gave a beige-orange solid (61%).

¹H NMR (300 MHz, CDCl₃, 293K, TMS): δ 9.07 (1H, s), 8.21 (1H, d, J=3 Hz), 7.91 (2H, d, J=4.8 Hz), 7.45 (1H, d, J=3 Hz), 7.34 (2H, d, J=5.1 Hz), 2.44 (3H, s).

¹³C NMR (75 MHz, CDCl₃, 293K, TMS): 153.6, 151.2, 143.0, 141.9, 129.3, 128.9, 126.9, 120.8, 21.6.

HRMS calculated for C₁₁H₁₀N₂O (M+) 186.0793; Found: 186.0780.

Melting point ° C.: 121.8-123.1.

IR (vₘₐₓ/cm⁻¹): 3076, 3035, 2922, 1371, 1255, 1038, 849, 808.

Rf (10% Acetone/DCM): 0.3

### Methyl 4-(pyrimidin-4-yl)benzoate N-oxide (Table 5, Entry 19)

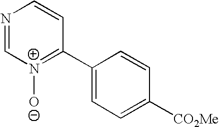

Synthesized according to general procedure 4. Purification via silica gel column chromatography using 100% DCM then a mixture of 20% Acetone/DCM gave a beige solid (50%).

¹H NMR (300 MHz, CDCl₃, 293K, TMS): δ 9.11 (1H, s), 8.28 (1H, d, J=5.1 Hz), 8.19 (2H, d, J=8.4 Hz), 8.06 (2H, d, J=8.1 Hz), 7.49 (1H, d, J=4.8 Hz), 3.97 (3H, s).

¹³C NMR (75 MHz, CDCl₃, 293K, TMS): 151.2, 143.3, 133.9, 132.3, 129.9, 129.7, 129.1, 121.2, 99.4, 52.5.

HRMS calculated for C₁₂H₁₀N₂O₃ (M+) 230.2194; Found: 230.0671.

IR (vₘₐₓ/cm⁻¹): 3029, 2920, 2857, 1733, 1652, 1254, 739.

Rf (20% Acetone/DCM): 0.3

### 2-(3-Methoxyphenyl)pyrimidine N-oxide (Table 5, Entry 18)

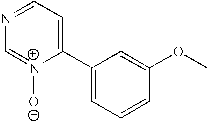

Synthesized according to general procedure 4. Purification via silica gel column chromatography using 100% DCM then a mixture of 20% Acetone/DCM gave an orange oil (62%).

¹H NMR (400 MHz, CDCl₃, 293K, TMS): δ 9.08 (1H, s), 8.23 (1H, d, J=4.8 Hz), 7.61 (1H, m), 7.46-7.41 (3H, m), 7.08 (1H, dt, J=2.4 and 9.6 Hz), 3.86 (3H, s).

¹³C NMR (100 MHz, CDCl₃, 293K, TMS): 159.4, 153.4, 151.1, 143.3, 130.9, 129.6, 121.2, 121.2, 117.2, 114.2, 55.4.

HRMS calculated for C₁₁H₁₀N₂O₂ (M+) 202.0742; Found: 202.0762.

IR (vₘₐₓ/cm⁻¹): 3080, 2962, 2837, 1696, 1585, 1477, 1258, 1028.

Rf (20% Acetone/DCM): 0.25

### 2-(3,5-dimethylphenyl)pyrimidine N-oxide (Table 5, Entry 17)

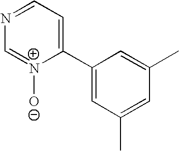

Synthesized according to general procedure 4. Purification via silica gel column chromatography using 100% DCM then a mixture of 15% Acetone/DCM gave a orange oil (55%).

¹H NMR (300 MHz, CDCl₃, 293K, TMS): δ 9.08 (1H, s), 8.22 (1H, d, J=4.8 Hz), 7.55 (2H, s), 7.42 (1H, d, J=5.1 Hz), 7.17 (1H, s), 2.39 (6H, s).

¹³C NMR (75 MHz, CDCl₃, 293K, TMS): 151.0, 143.2, 138.2, 132.9, 129.6, 126.6, 121.2, 21.3. 2 peaks are overlaping.

HRMS calculated for C₁₁H₁₂N₂O (M+) 200.0950; Found: 200.0970.

IR (vₘₐₓ/cm⁻¹): 3090, 2920, 2860, 1698, 1579, 1373, 1254, 834.

Rf (15% Acetone/DCM): 0.3

### Deoxygenated Aryidiazines

### 2-p-Tolylpyrazine (Table 6, Entry 1)

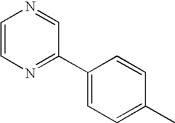

Synthesized according to general procedure 5. Purification via silica gel column chromatography using 100% DCM then a mixture of 2.5% Acetone/DCM gave a white solid (86%).

Exhibited identical spectral data according to previous reports²⁷.

### 2-(4-Methoxyphenyl)pyrazine (Table 6, Entry 2)

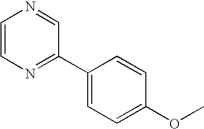

Synthesized according to general procedure 5. Purification via silica gel column chromatography using 100% DCM then a mixture of 5% Acetone/DCM gave a white solid (82%).

Exhibited identical spectral data according to previous reports²⁷.

### Methyl 4-(quinoxalin-2-yl)benzoate (Table 6, Entry 3)

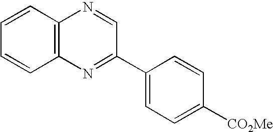

Synthesized according to general procedure 5. Purification via silica gel column chromatography using 100% DCM then a mixture of 10% Acetone/DCM gave a yellow solid (98%).

¹H NMR (300 MHz, CDCl₃, 293K, TMS): δ 9.35 (1H, s), 8.29-8.11 (6H, m), 7.84-7.72 (2H, m), 3.97 (3H, s).

¹³C NMR (75 MHz, CDCl₃, 293K, TMS): 166.5, 150.5, 143.1, 142.1, 141.7, 140.7, 131.3, 130.5, 130.2, 130.0, 129.7, 129.1, 127.4, 52.3.

HRMS calculated for C₁₆H₁₂N₂O₂ (M+) 264.0899; Found: 264.0883.

Melting point ° C.: 141.0-142.6.

IR (vₘₐₓ/cm⁻¹): 2952, 2924, 2853, 1733, 1605, 1285, 772, 755.

Rf (10% Acetone/DCM): 0.6

### 5,6,7,8-tetrahydro-2-p-Tolylquinoxaline (Table 6, Entry 4)

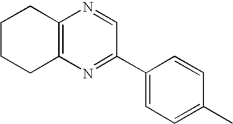

Synthesized according to general procedure 5. Purification via silica gel column chromatography using 100% DCM then a mixture of 3% Acetone/DCM gave a white solid (84%).

¹H NMR (300 MHz, CDCl₃, 293K, TMS): δ 8.71 (1H, s), 7.87 (2H, d, J=8.1 Hz), 7.29 (2H, d, J=8.1 Hz), 3.06-2.93 (4H, m), 2.41 (3H, s), 2.00-1.90 (4H, m).

¹³C NMR (75 MHz, CDCl₃, 293K, TMS): 152.1, 150.7, 149.7, 139.2, 138.6, 134.1, 129.6, 126.6, 32.2, 31.7, 22.7, 21.3. 2 peaks are overlaping.

HRMS calculated for C₁₅H₁₆N₂ (M+) 224.1313; Found: 224.1326.

Melting point ° C.: 80.0-81.2.

IR (vₘₐₓ/cm⁻¹): 3067, 3017, 2943, 2862, 1451, 1143, 826.

Rf (3% Acetone/DCM): 0.45

### 2,6-dip-tolylpyrazine (Table 6, Entry 5)

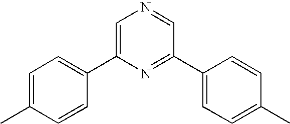

Synthesized according to general procedure 5. Purification via silica gel column chromatography using 100% DCM gave a white solid (76%).

Exhibited identical spectral data according to previous reports²⁸.

### 4-p-Tolylpyrimidine (Table 6, Entry 10)

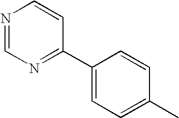

Synthesized according to general procedure 6. Purification via silica gel column chromatography using a mixture of 5% Acetone/DCM gave a beige solid (81%).

Exhibited identical spectral data according to previous reports²⁹.

### 3-Phenylpyridazine (Table 6, Entries 6 and 7)

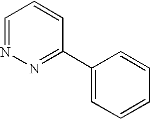

Synthesized according to general procedure 6. The product was obtained pure without purification (87%).

Exhibited identical spectral data according to previous reports³⁰.

### 3-o-Tolylpyridazine (Table 6, Entry 8)

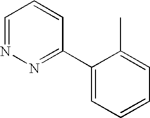

Synthesized according to general procedure 6. Purification via silica gel column chromatography using a mixture of 30% EtOAc/Benzene, then a mixture of 45% EtOAc/Benzene gave brown oil (70%).

¹H NMR (300 MHz, CDCl₃, 293K, TMS): δ 9.20 (1H, dd, J=1.8 and 6.0 Hz), 7.61-7.53 (2H, m), 7.46-7.31 (4H, m), 2.40 (3H, s).

¹³C NMR (75 MHz, CDCl₃, 293K, TMS): 162.2, 149.6, 137.2, 136.1, 130.9, 129.8, 129.2, 127.2, 126.1, 126.1, 20.3.

HRMS calculated for C₁₁H₁₀N₂ (M+) 170.0844; Found: 170.0838.

IR (vₘₐₓ/cm⁻¹): 3065, 2963, 2928, 1580, 1435, 765.

Rf (30% EtOAc/Benzene): 0.3

### 3-(4-Methoxyphenyl)pyridazine (Table 6, Entry 9)

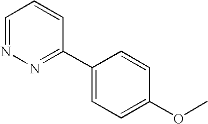

Synthesized according to general procedure 6. Purification via silica gel column chromatography using a mixture of 35% EtOAc/Benzene, then a mixture of 50% EtOAc/Benzene gave brown oil (70%).

¹H NMR (300 MHz, CDCl₃, 293K, TMS): δ 9.10 (1H, d, J=4.5 Hz), 8.06 (2H, d, J=9.0 Hz), 7.80 (1H, d, J=9.6 Hz), 7.49 (1H, dd, J=4.8 and 9.0 Hz), 7.05 (2H, d, J=8.7 Hz), 3.88 (3H, s).

¹³C NMR (75 MHz, CDCl₃, 293K, TMS): 161.3, 158.9, 149.4, 128.7, 128.4, 126.6, 123.1, 114.4, 55.3.

HRMS calculated for C₁₁H₁₀N₂O (M+) 186.0793; Found: 186.0794.

Melting point ° C.: 110.4-111.1.

IR (vₘₐₓ/cm⁻¹): 3054, 2929, 2847, 1612, 1436, 1249, 1025, 811.

Rf (30% EtOAc/benzene): 0.2

### Functionalized Diazine N-Oxides

### Methyl 4-(6-p-tolylpyrazin-2-yl)benzoate (102)

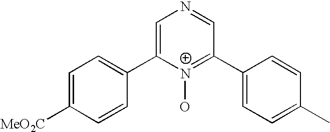

Synthesized according to general procedure 2. Purification via silica gel column chromatography using 15% Acetone/DCM then a mixture of 25% Acetone/DCM gave a beige solid (74%).

¹H NMR (300 MHz, CDCl₃, 293K, TMS): δ 8.58 (2H, d, J=10.8 Hz), 8.18 (2H, d, J=8.4 Hz), 7.93 (2H, d, J=8.4 Hz), 7.74 (2H, d, J=8.1 Hz), 7.33 (2H, d, J=8.1 Hz), 3.96 (3H,s), 2.43 (3H,s).

¹³C NMR (75 MHz, CDCl₃, 293K, TMS): 166.3, 146.9, 146.0, 144.8, 143.7, 140.7, 133.9, 131.4, 129.5, 129.4, 129.2, 129.2, 126.2, 52.3, 21.5.

HRMS calculated for C₁₉H₁₆N₂O₃ (M+) 320.1161; Found: 320.1141.

Melting point ° C.: 178.5-180.5.

IR (vₘₐₓ/cm⁻¹): 2964, 2921, 2854, 1727, 1612, 1291, 1105, 815.

Rf (10% Acetone/DCM): 0.15

### 2-Chloro-6-p-tolylpyrazine (103)

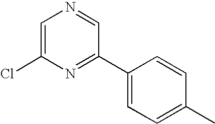

To a solution of toluene (1.0 mL) and DMF (1.0 mL) was added POCl₃ (0.049 mL, 0.54 mmol). The mixture was stirred for 10 minutes at 0° C., then 2-p-tolylpyrazine N-oxide (50 mg, 0.27 mmol) in DMF (0.5 mL) was added. After 10 minutes, the reaction mixture was allowed to warm to room temperature and stirred over night. The solvent was then evaporated via Kugelrohr distillation. The residue was cooled in a ice bath and a saturated solution of NaHCO₃ was added. The aqueous layer was extracted 3 times with DCM. The combined organic phases was dried over MgSO₄, filtered and concentrated under vacuum to give a pure pale yellow solid (54 mg, 98%).

¹H NMR (300 MHz, CDCl₃, 293K, TMS): δ 8.89 (1H, s), 8.47 (1H, s), 7.92 (2H, d, J=7.8 Hz), 7.31 (2H, d, J=7.8 Hz), 2.42 (3H, s).

¹³C NMR (75 MHz, CDCl₃, 293K, TMS): 152.5, 148.8, 141.9, 140.9, 139.1, 131.9, 129.8, 126.9, 21.4.

HRMS calculated for C₁₁H₉N₂Cl (M+) 204.0454; Found: 204.0447.

Melting point ° C.: 72.5-73.7.

IR (vₘₐₓ/cm⁻¹): 3029, 2919, 2855, 1507, 1158, 1005, 821.

Rf (10% Acetone/DCM): 0.5

### 2-Chloro-6-(4-methoxyphenyl)pyrazine

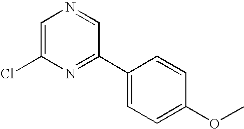

To a solution of toluene (1.0 mL) and DMF (1.0 mL) was added POCl₃ (0.045 mL, 0.49 mmol). The mixture was stirred for 10 minutes at 0° C., then 2-(4-methoxyphenyl)pyrazine N-oxide (50 mg, 0.25 mmol) in DMF (0.5 mL) was added. After 10 minutes, the reaction mixture was allowed to warm to room temperature and stirred over night. The solvent was then evaporated via Kugelrohr distillation. The residue was cooled in a ice bath and a saturated solution of NaHCO₃ was added. The aqueous layer was extracted 3 times with DCM. The combined organic phases was dried over MgSO₄, filtered and concentrated under vacuum to give a pure pale yellow solid (47.3 mg, 87%). Exhibited identical spectral data according to previous reports³¹.

### 2-p-Tolylpiperazine (106)

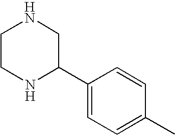

To a round bottom flask was added Pt₂O (8 mg, 0.03 mmol) and 2-p-tolyl-pyrazine N-oxide (50 mg, 0.27 mmol). The mixture was then purged under nitrogen for 10 minutes. Addition of the acetic acid (3 mL) was followed by the addition of hydrogen via a balloon. When complete, the reaction mixture was filtered trought a pad of celite, and the solvent was evaporated via Kugelrohr distillation. A 10% solution of NaOH was added and the aqueous layer was extracted 3 times with DCM. The combined organic phases was dried over MgSO₄, filtered and concentrated under vacuum to give a pure beige solid (32 mg, 68%).

¹H NMR (300 MHz, CDCl₃, 293K, TMS): δ 7.27 (2H, d, J=7.8 Hz), 7.13 (2H, J=7.8 Hz), 3.71 (1H, dd, J=2.4 and 9 Hz), 3.13-2.82 (5H, m), 2.70 (1H, dd, J=10.2 and 12 Hz), 2.33 (3H, s), 1.84 (2H,s).

¹³ C NMR (75 MHz, CDCl₃, 293K, TMS): 139.7, 137.0, 129.0, 126.7, 61.7, 54.3, 47.8, 46.0, 21.0.

HRMS calculated for C₁₁H₆N₂ (M+) 176.1313; Found: 176.1319.

Melting point ° C.: 88.7-90.3.

IR (vₘₐₓ/cm⁻¹): 3274, 3018, 2940, 2826, 1514, 813.

Rf (10% Acetone/DCM): 0.05

### 2-Morpholino-6-p-tolyl pyrazine (104)

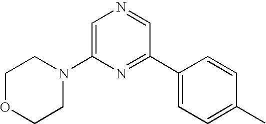

To a dry Schlenck tube was added 2-chloro-6-p-tolylpyrazine (60 mg, 0.29 mmol), sodium tert-butoxide (40 mg, 0.41 mmol), Pd(OAc)₂ (2 mg, 0.01 mmol) and 2-(dicyclohexylphosphino) biphenyl (6 mg, 0.02). The mixture was then purged under nitrogen for 10 minutes. Addition of morpholine (0.031 mL, 0.35 mmol) was followed by the addition of degassed toluene (1.0 mL). The reaction mixture was heated at 100° C. over night. The reaction was filtered trought a pad of celite, and the solvent was evaporated under reduce pressure. Purification of the residue via silica gel column chromatography using 100% DCM, then a mixture of 5% Acetone/DCM gave a pale yellow solid (70%).

¹H NMR (300 MHz, CDCl₃, 293K, TMS): δ 8.37 (1H, s), 8.03 (1H, s), 7.90 (2H, d, J=8.1 Hz), 7.27 (2H, d, J=8.1 Hz), 3.86 (4H, t, J=4.5 Hz), 3.65 (4H, t, J=5.1 Hz), 2.40 (3H,s).

¹³C NMR (75 MHz, CDCl₃, 293K, TMS): 154.1, 149.3, 139.5, 134.1, 130.2, 129.4, 128.3, 126.6, 66.6, 44.7, 21.3.

HRMS calculated for C₁₅H₁₇N₃O (M+) 255.1372; Found: 255.1362.

Melting point ° C.: 88.9-90.1.

IR (vₘₐₓ/cm⁻¹ ): 3058, 2963, 2854, 1525, 1253, 820.

Rf (5% Acetone/DCM): 0.3

### 2-Ethoxy-6-p-tolylpyrazine (105)

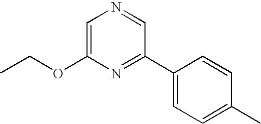

To a round bottom flask was added 2-chloro-6-p-tolylpyrazine (60 mg, 0.29 mmol), sodium ethoxide (60 mg, 0.88 mmol) and EtOH (3 mL). The reaction mixture was heated at 90° C. for 2 days. The solvent was evaporated under reduce pressure and the residue was extracted 3 times using water/brine and DCM. The combined organic phases was dried over MgSO₄, filtered and concentrated under vacuum. Purification of the residue via silica gel column chromatography using 100% DCM, then a mixture of 2% Acetone/DCM gave a pale yellow solid (85%).

¹H NMR (300 MHz, CDCl₃, 293K, TMS): δ 8.55 (1H, s), 8.10 (1H, s), 7.92 (2H, d, J=7.8 Hz), 7.28 (2H, d, J=7.8 Hz), 4.50 (2H, q, J=6.9 Hz), 2.41 (3H, s), 1.45 (3H, t, J=6.9 Hz).

¹³C NMR (75 MHz, CDCl₃, 293K, TMS): 159.4, 148.9, 139.7, 133.5, 133.2, 132.6, 129.5, 126.6, 61.9, 21.3, 14.4.

HRMS calculated for C₁₃H₁₄N₂O (M+) 214.1106; Found: 214.1115.

Melting point ° C.: 61.5-62.8.

IR (vₘₐₓ/cm⁻¹): 3070, 2981, 2920, 1538, 1424, 826.

Rf (2% Acetone/DCM): 0.3

### 6-p-Tolylpyrazin-2-yl acetate (101)

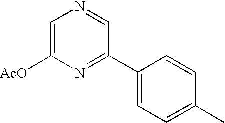

To a round bottom flask was added 2-p-tolylpyrazine N-Oxide and acetic anhydride (0.65 mL). The solvent was evaporated via Kugelrohr distillation and the residue was stirred over night at 50° C. in a acetone/silica gel mixture. The solvent was removed under vacum purified via silica gel column chromatography using a mixture of 20% Acetone/DCM. A yellow oil was obtained (71%).

¹H NMR (300 MHz, CDCl₃, 293K, TMS): δ 8.92 (1H, s), 8.39 (1H, s), 7.90 (2H, d, J=8.1 Hz), 7.30 (2H, d, J=8.1 Hz), 2.41 (6H, s).

¹³C NMR (75 MHz, CDCl₃, 293K, TMS): 168.5, 153.8, 151.4, 140.6, 139.0, 136.2, 132.2, 129.8, 127.0, 21.4, 21.1.

HRMS calculated for C₁₃H₁₂N₂O₂ (M+) 228.0899; Found: 228.0880.

IR (vₘₐₓ/cm⁻¹): 3062, 2924, 2855, 1773, 1531, 1185, 822.

Rf (20% Acetone/DCM): 0.3

### REFERENCES

¹ (a) Metal-catalyzed Cross-coupling Reactions; Diederich, F., Stang, P. J., Eds.; Wiley-VCH: New York, 1998. (b) Hassan, J.; Sévignon, M.; Gozzi, C.; Shulz, E.; Lemaire, M. Chem. Rev. 2002, 102, 1359.

² Littke, A. F.; Dai, C.; Fu, G. C. J. Am. Chem. Soc. 2000, 122, 4020.

³ (a) Molander, G. A.; Biolatto, B. J. Org. Chem. 2003, 68, 4302 and references therein. (b) Hodgson, P. B.; Salingue, F. H. Tetrahedron Lett. 2004, 45, 685.

⁴ Fang, A. G.; Mello, J. V.; Finney, N. S. Org. Lett. 2003, 5, 967.

⁵ (a) Davies, I. W.; Marcoux, J. F.; Reider, P. J. Org. Lett. 2001, 3, 209. (b) Roppe, J. R.; Wang, B.; Huang, D.; Tehrani, L.; Kamenecka, T.; Schweiger, E. J.; Anderson, J. J.; Brodkin, J.; Jiang, X.; Cramer, M.; Chung, J.; Reyes-Manalo, G.; Munoz, B.; Cosford, N. D. P. Bioorg. Med. Chem. Lett. 2004, 14, 3993.

⁶ (a) Kakiuchi, F.; Murai, S. Acc. Chem. Res. 2002, 35, 826. (b) Ritleng, V.; Sirlin, C.; Pfeffer, M. Chem. ReV. 2002, 102, 1731. (c) Miura, M.; Nomura, M. Top. Curr. Chem. 2002, 219, 211. (d) Kakiuchi, F.; Chatani, N. AdV. Synth. Catal. 2003, 345, 1077.

⁷ (a) Wang, X.; Lane, B. S.; Sames, D. J. Am. Chem. Soc. 2005, 127, 4996. (b) Lewis, J. C.; Wiedemann, S. H.; Bergmann, R. G.; Ellman, J. A. Org. Lett. 2004, 6, 35. (c) Park, C.-H.; Ryabova, V.; Seregin, I. V.; Sromek, A. W.; Gevorgyan, V. Org. Lett. 2004, 6, 1159. (d) Li, W.; Nelson, D. P.; Jensen, M. S.; Hoerrner, R. S.; Javadi, G. J.; Cai, D.; Larsen, R. D. Org. Lett. 2003, 5, 4835. (e) Okazawa, T.; Satoh, T.; Miura, M.; Nomura, M. J. Am. Chem. Soc. 2002, 124, 5286. (f) McClure, M. S.; Glover, B.; McSorley, E.; Millar, A.; Osterhout, M. H.; Roschangar, F. Org. Lett. 2001, 3, 1677.

⁸ (a) Kalyani, D.; Deprez, N. R.; Desai, L. V.; Sanford, M. S. J. Am. Chem. Soc. 2005, 127, 7330 and references therein. (b) Daugulis, O.; Zaitsev, V. G. Angew. Chem., Int. Ed. 2005, 44, 2.

⁹ (a) Campeau, L.-C.; Parisien, M.; Leblanc, M.; Fagnou, K. J. Am. Chem. Soc. 2004, 126, 9186. (b) Campeau, L.-C.; Parisien, M.; Jean, A.; Fagnou, K. J. Am. Chem. Soc. 2005, accepted. (c) Huang, Q.; Fazio, A.; Dai, G.; Campo, M. A.; Larock, R. C. J. Am. Chem. Soc. 2004, 126, 7460 and references therein.

¹⁰ (a) Godula, K.; Sezen, B.; Sames, D. J. Am. Chem. Soc. 2005, 127, 3648. (b) Mukhopadhyay, S.; Rothenberg, G.; Gitis, D.; Baidossi, M.; Ponde, D. E.; Sasson, Y. J. Chem. Soc., Perkin Trans. 2 2000, 1809-1812.

¹¹ (a) Metal-catalyzed Cross-coupling Reactions; Diederich, F., Stang, P. J., Eds.; Wiley-VCH: New York, 1998. (b) Hassan, J.; Sevignon, M.; Gozzi, C.; Shulz, E.; Lemaire, M. Chem. Rev. 2002, 102, 1359

¹² (a) A. F. Pozharski, A. T. Soldartenko, A. Katritsky, Heterocycles in Life and Society, Wiley, New York, 1997.

¹³ N. Kudo, M.; Perseghini, G. C. Fu Angew. Chem. Int. Ed. 2006, 45, 1282-1284.

¹⁴ K. L. Billingsley, K. W. Anderson, S. L. Buchwald Angew. Chem. Int. Ed. 2006, 45, 3484-3488.

¹⁵ For example, a number of pinacol boronic esters of the type illustrated in Scheme 1 are commercially available from Combiphos Catalysts, Inc. for approximately $1000USD per gram (http://www.combiphos.com).

¹⁶ (a) Murray, R. W.; Jeyaraman, R. J. Org. Chem. 1985, 50, 2847. (b) van den Heuvel, M.; van den Berg, T. A.; Kellogg, R. M.; Choma, C. T.; Feringa, B. L. J. Org. Chem. 2004, 69, 250.

¹⁷ Ando, T.; Fujimoto, Y.; Morisaki, S. J. Haz. Mater. 1991, 28, 251.

¹⁸ (a) Balicki, R. Synthesis 1989, 8, 645. (b) Aoyagi, Y.; Abe, T.; Ohta, A. Synthesis 1997, 8, 891.

¹⁹ (a) Kakiuchi, F.; Murai, S. Acc. Chem. Res. 2002, 35, 826. (b) Ritleng, V.; Sirlin, C.; Pfeffer, M. Chem. Rev. 2002, 102, 1731. (c) Miura, M.; Nomura, M. Top. Curr. Chem. 2002, 219, 211. (d) Kakiuchi, F.; Chatani, N. Adv. Synth. Catal. 2003, 345, 1077. (e) L.-C. Campeau, K. Fagnou, Chem. Commun. 2005, 1253.

²⁰ L. C. Campeau, S. Rousseaux, K. Fagnou J. Am. Chem. Soc. 2005, 127, 18020.

²¹ T. Choshi, Y. Matsuya, M. Okita, K. Inada, E. Sugino, S. Hibino, Tetrahedron. Lett. 1998, 39, 2341.

²² van Galen, P. J. M. et. al. J. Med. Chem.1991, 34, 1202.

²³ J. P. Wolfe, H. Tomori, J. P. Sadighi, J. Yin, S. L. Buchwald, J. Org. Chem. 2005, 65, 1158.

²⁴ Arthur F. Kluge, Michael L. Maddox, Graham S. Lewis J. Org. Chem., 1980, 45(10), 1909-1914.

²⁵ A. Ohta et. al. J. Heterocycl. Chem., 1982, 19, 465-473.

²⁶ M. B. Mitchell, P. J. Wallbank Tet. Lett., 1991, 32(20), 2273.

²⁷ D. H. Huh, H. Ryu and Y. G. Kim Tetrahedron, 2004, 60, 9857-9862.

²⁸ G. Jia, Z. Lim and Y. Zhang Heteroatom Chemistry, 1998, 9(3), 3341-345.

²⁹ S. N. Balasubrahmanyam, B. Jeyashri and I. N. N. Namboothiri Tetrahedron, 1994, 50(27), 8127-8142.

³⁰ M. S. South, T. L. Jakuboski, M. D. Westmeyer and D. R. Dukesherer J. Org. Chem., 1996, 61(25), 8921-8934.

³¹ F. Buron, N. Ple, A. Turck and G. Queguiner J. Org. Chem., 2005, 70(7), 2616.

1. A coupling process comprising:
(i) reacting a compound of general formula 1 with a compound of general formula A-X to obtain a compound of general formula 2; or
(ii) reacting a compound of general formula 2A with a compound of general formula A′-X, to obtain a compound of general formula 4, 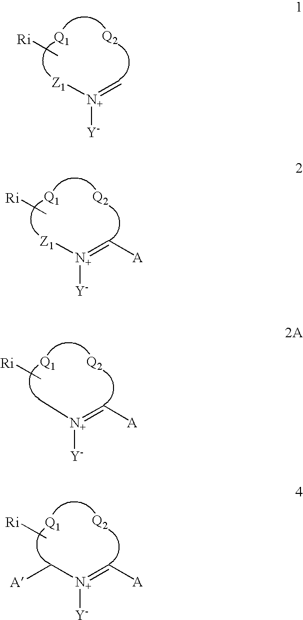
wherein the reaction in (i) or (ii) takes place in the presence of a first metal catalyst, and wherein:
Y is O or S;
Z₁ is C, N, O or S, and is optionally substituted when it is C or N;
Q₁, Q₂ and A each represents a chemical group which is independently linear or branched, saturated or unsaturated, aromatic, cyclic, bicyclic or biaryl the chemical group containing or not containing a hetero atom which is N, O, S or a halogen atom;
( denotes a chemical bond that is present or absent;
Ri represents at least one substituent that is linear or branched, saturated or unsaturated, aromatic, cyclic or bicyclic, the substituent containing or not containing a hetero atom, with the proviso that N, Z₁, Q₁, Q₂ and C form a ring, optionally Ri together with the ring forms a bicyclic or biaryl group;
X represents a leaving group;
C directly attached to N⁺ in 1 is not substituted; and
the other C directly attached to N+ and not bearing substituent A in 2A is not substituted.

2. A process according to claim 1 further comprising:
(iii) converting the compound of general formula 2 to a compound of general formula 3; or
(iv) converting the compound of general formula 4 to a compound of general formula 5, 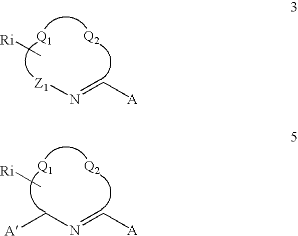
wherein the reaction in (iii) or (iv) takes place in the presence of a second metal catalyst, and wherein all the groups and substituents are defined in claim 1.

3. A coupling process comprising:
(i) reacting a compound of general formula 6 with a compound of general formula 29, to obtain a compound of general formula 7; or
(ii) reacting a compound of general formula 7A with a compound of general formula 30, to obtain a compound of general formula 9; or
(iii) reacting a compound of general formula 6′ with a compound of general formula 29, to obtain a compound of general formula 7′; or
(iv) reacting a compound of general formula 7A′ with a compound of general formula 30, to obtain a compound of general formula 9′; 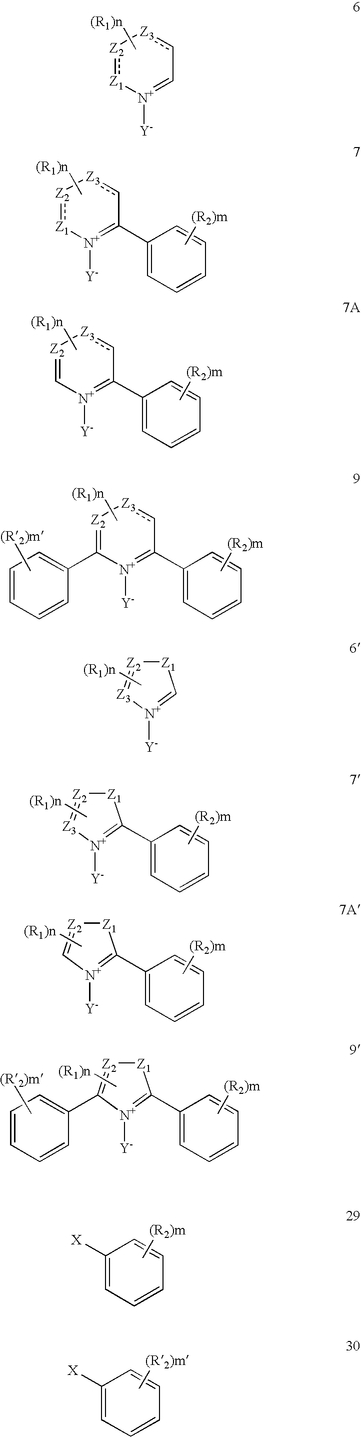
wherein the reaction in (i), (ii), (iii) or (iv) takes place in the presence of a first metal catalyst, and wherein:
Y is O or S;
Z₁, Z₂ and Z₃ are each independently C, N, O or S, and are each independently optionally substituted when they are C or N;
R₁ and R₂ are each independently linear or branched, saturated or unsaturated, aromatic, cyclic, bicyclic, contains or not contains a hetero atom which is N, O, S or a halogen atom, optionally R₁ or R₂ together with the ring to which it is attached forms a bicyclic or biaryl group;
— denotes a chemical bond that is present or absent;
n is 0, 1, 2, 3 or 4;
m is 0, 1, 2, 3, 4 or 5;
X is a leaving group;
C directly attached to N⁺ in 6 is not substituted;
C directly attached to N⁺ in 6′ is not substituted; and
the other C directly attached to N+ and not bearing the phenyl group in 7A′ is not substituted.

4. A process according to claim 3 further comprising:
(v) converting the compound of general formula 7 to a compound of general formula 8; or
(vi) converting the compound of general formula 9 to a compound of general formula 10; or
(vii) converting the compound of general formula 7′ to a compound of general formula 8′; or
(viii) converting the compound of general formula 9′ to a compound of general formula 10′; 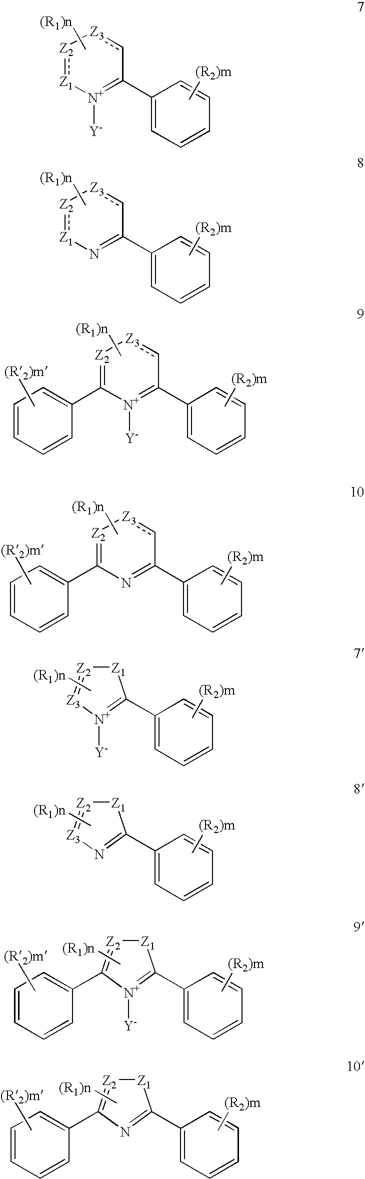
wherein the reaction in (v), (vi), (vii) or (viii) take place in the presence of a second metal catalyst, and wherein all the groups and substituents are as defined in claim 3.

5. A process according to claim 3, wherein:
(a) the compound of general formula 6 is selected from 11, 16, 19 and 24; 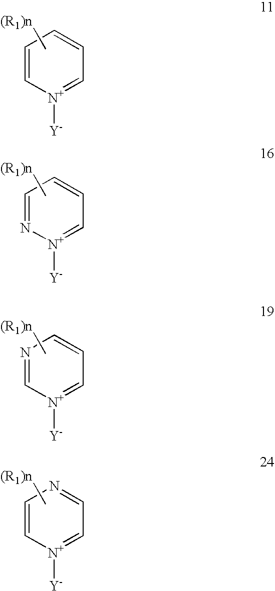
(b) the compound of general formula 7A is selected from 12, 17, 20 and 25; 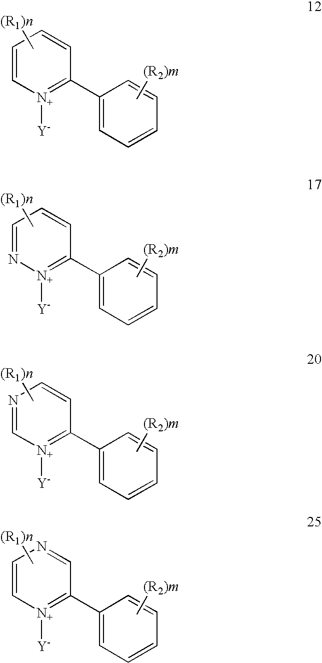
(c) the compound of general formula 6′ is selected from 32, 33 and 34; and 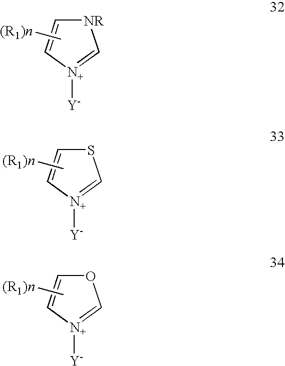
(d) the compound of general formula 7A′ is selected from 35, 36 and 37 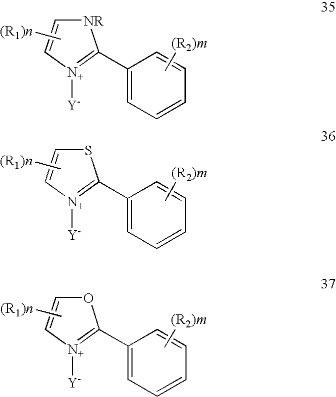
wherein R is H, alkyl or aryl.

6. A process according to claim 1 or 3, wherein the first metal catalyst is a transition metal catalyst which is selected from Pd(OAc)₂, PdCl₂, PdBr₂ and PdI₂.

7. A process according to claim 1 or 3, wherein the reaction takes place in the presence of a metal salt which is selected from CuCN, CuCl, CuBr and CuI; the metal salt being used in an amount of about 1 to 15 mol % based on the compound of general formula A-X or A′-X′.

8. A process according to claim 1 or 3, wherein the reaction takes place in the presence of a base which is selected from K₂CO₃, NaOH, KOH and K₃PO₄; the base being used in an equivalent amount of about 1 to 4 of the base based on the compound of general formula A-X or A′-X.

9. A process according to claim 1 or 3, wherein the reaction takes place at a temperature of about 80 to 130° C.

10. A process according to claim 1 or 3, wherein the reaction takes place in the presence of an organic solvent which is an aromatic solvent, dioxane, mesitylene, N,N-dimethylacetamide, N,N-dimethylformamide, N-methylpyrrolidinone, tetrahydrofuran, dichloromethane, ether or a mixture thereof.

11. A process according to claim 1 or 3, wherein the reaction takes place in the presence of a phosphorous donor ligand or a N-heterocyclic carbene ligand, the ligand being used in an amount of about 10 to 20 mol % based on the compound of general formula A-X or A′-X.

12. A process according to claim 1 or 3, wherein the reaction takes place in the presence of an additive which is capable of overcoming the poisoning effects of N-oxide substrates, the additive being used in an equivalent amount of about 0.1 to 4 based on the compound of general formula A-X or A′-X.

13. A process according to claim 1, wherein an equivalent amount of about 1 to 6 of the compound of general formula 1 based on the compound of general formula A-X, is used; and an equivalent amount of about 1 to 6 of the compound of general formula 2A based on the compound of general formula A′-X, is used.

14. A process according to claim 1, wherein an amount of about 2 to 10 mol % of the first metal catalyst based on the compound of general formula A-X, is used; and an amount of about 2 to 10 mol % of the first metal catalyst based on the compound of general formula A′-X, is used.

15. A process according to claim 1 or 3, wherein the reaction time is about 5 to 30 hours.

16. A process according to claim 1 or 3, wherein the leaving group is a halogen atom or a sulfonate group.

17. A process according to claim 1 or 3, wherein the substitution is regioselective to a carbon atom attached to N⁺.

18. A process according to claim 2 or 4, wherein the second metal catalyst is a hydrogenation catalyst comprising Pd, Pt, Rh, Ir or Rn.

19. A process according to claim 2 or 4, wherein the conversion takes place in the presence of an organic salt or a gas.

20. A process according to claim 2 or 4, wherein the conversion takes place in the presence of an organic solvent which is MeOH, EtOH, iPrOH, EtOAc, THF, acetone or a mixture thereof.

21. A process according to claim 2 or 4, wherein the conversion takes place at a temperature of about 15 to 30° C.

22. A process according to claim 1 further comprising using the compounds of general formulae 1, 1A or 4 in the preparation of target compounds of therapeutic or industrial value.
